(19) United States
(12) Patent Application Publication
(10) Pub. No.: US 2019/0300849 A1
(43) Pub. Date: Oct. 3, 2019

(54) STEM/PROGENITOR CELLS FROM DUODENAL BRUNNER'S GLANDS AND METHODS OF ISOLATING AND USING THEM

(71) Applicants: The University of North Carolina at Chapel Hill, Chapel Hill, NC (US); Sapienza Universitá di Roma, Rome, (IT)

(72) Inventors: Guido Carpino, Rome (IT); Vincenzo Cardinale, Rome (IT); Lola Reid, Chapel Hill, NC (US); Domenico Alvaro, Rome (IT); Eugenio Gaudio, Rome (IT)

(73) Assignees: The University of North Carolina at Chapel Hill, Chapel Hill, NC (US); Sapienza Universitá di Roma, Rome, (IT)

(21) Appl. No.: 16/368,524

(22) Filed: Mar. 28, 2019

Related U.S. Application Data
(60) Provisional application No. 62/650,208, filed on Mar. 29, 2018.

(51) Int. Cl. C12N 5/071; C12N 5/00; A61K 35/407

(52) U.S. Cl. CPC C12N 5/067; C12N 5/0676; A61K 35/407; C12N 5/0018

(57) ABSTRACT

Disclosed herein is Brunner's Gland Stem/Progenitor Cells (BGSCs) having phenotypic traits of endodermal stem cells and positive for pluripotency markers, and methods of isolating them from the human duodenum. Moreover, the present disclosure provides that BGSCs are easily isolated from duodenum from human donors, can be expanded in culture or induced to differentiate towards hepatic and pancreatic lineages and could represent a cell source for clinical programs of regenerative medicine.

### CROSS-REFERENCE TO RELATED PATENT APPLICATIONS

This application claims priority from U.S. Provisional Patent Application No. 62/650,208, filed Mar. 29, 2018, incorporated herein by reference in its entirety.

### BACKGROUND

Multiple stem/progenitor cell niches persist in specific anatomical locations within the fetal and postnatal human biliary tree. Although stem/progenitor cell populations have long been recognized in fetal tissues, their persistence in adult tissues is newly recognized. Glandular elements, peribiliary glands (PBGs), with parallels to intestinal crypts are located within extrahepatic bile ducts, large intrahepatic bile ducts, and in the hepato-pancreatic common duct; later stage stem/progenitor cells and derived from those in PBGs are found within the gallbladder. The network continues from the stem/progenitors in the PBGs of the hepato-pancreatic common duct to committed progenitors within pancreatic duct glands (PDGs) within the pancreas. The stem/progenitors in all of these niches are collectively termed Biliary Tree Stem/progenitor Cells (BTSCs). BTSCs within PBGs have traits of endodermal stem/progenitor cells including proliferative capabilities, self-renewal and multipotency; they have traits of progenitors in PDGs that include proliferative capabilities and multipotency but less self-replicative ability than of the stem/progenitor cells in the PBGs.

BTSCs located at the level of the hepato-pancreatic ampulla are primitive, co-express several pluripotency markers (e.g. OCT4, SOX2, NANOG), can self-renew or differentiate into functional hepatocytes, cholangiocytes and pancreatic islets (whether they can also give rise to acinar cells is being considered in ongoing studies). Niches containing BTSCs extend into the liver and into the pancreas. Detailed anatomical studies in humans have revealed a proximal-to-distal axis for the BTSC niche organization going from the proximal site, the hepato-pancreatic ampulla, where the most primitive stem cells are located, to the distal site, the liver or the pancreas, where mature cells are found. This axis recapitulates the organogenesis of these organs and reflects their common embryological origin. Indeed, from an embryological point of view, the common precursors for liver, bile duct system and pancreas exist at early stages of development in the definitive ventral endoderm forming the foregut. At this stage of development, the primitive duodenum harbors ventral endodermal stem/progenitor cells.

The most primitive stem/progenitors identified are those found within Brunner's Glands, located within the submucosa of the duodenum. These cells may be the start-point of the entire network of stem/progenitor niches giving rise to liver and to pancreas. Isolation of these cells from adult duodena have been difficult and has not been achieved by methods disclosed in the art to date. The practical significance of these cells is manifold. For example, these cells can be used for in vitro assessments of drug effects, and for generating model systems (e.g. organoids) for analyses of liver and pancreatic development, function, maintenance and/or repair (given that these contain the precursors to both organs), for other clinical or analytical tests pertinent to liver, pancreas and other endodermal tissue, and for diagnosis or treatment of diseases or conditions involving or affecting the liver, pancreas and/or other endodermal tissue. The cells from Brunner's Glands are unique among the sources of endodermal stem/progenitor cells in being in a location, the duodenum, accessible by endoscopy and so useful for sourcing of stem/progenitor cells for autologous or heterologous cell therapies or gene therapies. In addition, tumors derived from these Brunner's Glands are logical targets for various forms of cancer therapies. Thus, there remains a need in the art to develop methods to isolate the cells of interest, known as “Brunner's Gland stem/progenitor cells.”

### SUMMARY

In one aspect, the present disclosure relates to a stem/progenitor cell, isolated from a duodenum (referred to as a Brunner's Gland stem/progenitor cell or BGSC), which expresses one or more of the markers selected from the group consisting of Tra-1-60, Tra-1-81, OCT4, SOX2, NANOG, EpCAM, SOX9 and cytokeratin 7 (CK7) and which is further characterized as capable of proliferation, with limited or minimal differentiation, under culture conditions that support self-renewal.

In another aspect, the present disclosure relates to a stem/progenitor cell isolated from a duodenum (referred to as a Brunner's Gland stem/progenitor cell or BGSC), which expresses one or more of the markers selected from the group consisting of Lgr5, NIS, CD44 and CK19 and which is further characterized as capable of proliferation, with limited or minimal differentiation, under culture conditions that support self-renewal.

In another aspect, the present disclosure relates to a stem/progenitor cell isolated from a duodenum (referred to as a Brunner's Gland stem/progenitor cell or BGSC), which expresses both SOX17 and PDX1 and which is further characterized as capable of proliferation, with limited or minimal differentiation, under culture conditions that support self-renewal.

In another aspect, the present disclosure relates to a stem/progenitor cell isolated from a duodenum (referred to as a Brunner's Gland stem/progenitor cell or BGSC), which expresses one or more of the markers selected from the group consisting of Tra-1-60, Tra-1-81, OCT4, SOX2, NANOG, EpCAM, SOX9, CK7, Lgr5, NIS, CD44 and CK19, or which expresses both SOX17 and PDX1, and which is further characterized as capable of proliferation, with limited or minimal differentiation, under culture conditions that support self-renewal.

In some embodiments, the BGSC is substantially free of pathogens and/or pathogenic and/or beneficial microbes. In some embodiments, the BGSC can be proliferated, with limited or minimal differentiation, for at least one month. In some embodiments, the BGSC can be proliferated, with limited or minimal differentiation, for at least two months. In some embodiments, the BGSC can be proliferated, with limited or minimal differentiation, for at least six months. In some embodiments, the BGSC can be proliferated, with limited or minimal differentiation, for at least twelve months.

In some embodiments, the culture conditions that support self-renewal of the BGSC comprise a serum-free medium, optionally Kubota's Medium. In some embodiments, the culture conditions that support self-renewal comprise a medium containing serum.

In one aspect, the present disclosure relates to a population of stem/progenitor cells isolated from duodenum, in which at least some, or a substantial portion of, or a majority of the cells expresses one or more of the markers selected from the group consisting of Tra-1-60, Tra-1-81, OCT4, SOX2, NANOG, EpCAM, SOX9 and CK7.

In one aspect, the present disclosure relates to a population of stem/progenitor cells isolated from duodenum, in which at least some, or a substantial portion of, or a majority of the cells expresses one or more of the markers selected from the group consisting of Lgr5, NIS, CD44 and CK19.

In one aspect, the present disclosure relates to a population of stem/progenitor cells isolated from duodenum, in which at least some, or a substantial portion of, or a majority of the cells expresses both SOX17 and PDX1.

In one aspect, the present disclosure relates to a population of stem/progenitor cells isolated from duodenum, in which at least some, or a substantial portion of, or a majority of the cells expresses one or more of the markers selected from the group consisting of Tra-1-60, Tra-1-81, OCT4, SOX2, NANOG, EpCAM, SOX9, CK7, Lgr5, NIS, CD44 and CK19, or in which at least some, or a substantial portion of, or a majority of the cells expresses both SOX17 and PDX1.

In some embodiments, the population of stem/progenitor cells is substantially free of pathogens and/or pathogenic and/or beneficial microbes.

In some embodiments, the population of stem/progenitor cells can be proliferated, with limited or minimal differentiation, for at least one month. In some embodiments, the population of stem/progenitor cells can be proliferated, with limited or minimal differentiation, for at least two months. In some embodiments, the population of stem/progenitor cells can be proliferated, with limited or minimal differentiation, for at least six months. In some embodiments, the population of stem/progenitor cells can be proliferated, with limited or minimal differentiation, for at least twelve months.

In some embodiments, the culture conditions that support self-renewal of the population of stem/progenitor cells comprise a serum-free medium, optionally Kubota's Medium. In some embodiments, the culture conditions that support self-renewal of the population of stem/progenitor cells comprise a medium containing serum.

In one aspect, the present disclosure relates to a method of isolating one or more BGSCs, or the a population of BGSCs from a duodenum of a subject, a portion thereof, or a sample taken from same comprising:
contacting a mucosal layer of a duodenum, which is substantially free of intestinal mucus, with a medium or solution having osmolality properties falling outside a physiological range under conditions that induce osmotic shock to the cells of the mucosal layer;
removing or dissolving at least a portion of the mucosal layer or the cells thereof by mechanical, surgical and/or chemical methods, leaving and/or exposing a remainder which may include a submucosal layer;
digesting or dissociating the remainder; and
isolating one or more BGSC or the population of BGSC from the digested remainder.

In some embodiments, the isolating step comprises isolating BGSCs which express, or a population of BGSCs in which at least some, or a substantial portion of, or a majority of the cells expresses, one or more markers selected from the group Tra-1-60, Tra-1-81, OCT4, SOX2, NANOG, EpCAM, SOX9, CK7, Lgr5, NIS, CD44, and CK19.

In some embodiments, the isolating step comprises isolating BGSCs, which express, or a population of BGSCs in which at least some, or a substantial portion of, or a majority of the cells expresses, both SOX17 and PDX1.

In one aspect, the present disclosure relates to a method of isolating one or more BGSCs, or the population of BGSCs from a duodenum of a subject, a portion thereof, or a sample taken from same, comprising the following steps, in which the step to substantially kill, inactivate, or remove pathogens and/or pathogenic and/or beneficial microbes can be carried out at any time or more than once:
removal of the intestinal mucus;
applying a medium or solution having osmolality properties falling outside a physiological range under conditions under conditions that induce osmotic shock to the cells of the mucosal layer;
removing or dissolving at least a portion of the mucosal layer or the cells thereof by mechanical, surgical and/or chemical methods, leaving and/or exposing a remainder which may include a submucosal layer;
applying to the mucosal layer and/or the remainder a medium or solution to substantially kill, inactivate or remove pathogens and/or pathogenic and/or beneficial microbes;
applying digestion or dissociation to the submucosal layer to produce a digest, dissociated cellular material, or a cell suspension;
optionally culturing at least some of the digest, dissociated cellular material, or cells from the cell suspension; and
isolating those cells that express, or a population of cells in which at least some, or a substantial portion of, or a majority of the cells express, one or more of Tra-1-60, Tra-1-81, OCT4, SOX2, NANOG, EpCAM, SOX9, CK7, Lgr5, NIS, CD44, CK19; and/or those cells that express both SOX17 and PDX1.

In some embodiments, the removal of intestinal mucus comprises squeezing the duodenum tissue.

In some embodiments, the medium or solution having osmolality properties falling outside a physiological range comprises a hypotonic, hypoosmotic, hypertonic, or hyperosmotic solution.

In some embodiments, the medium or solution having osmolality properties falling outside a physiological range comprises a glucose solution, a high salt solution, or distilled water.

In some embodiments, the removal is carried out by chemical disruption which comprises a use of an emulsifier and/or a detergent.

In some embodiments, the detergent and/or emulsifier is in water, saline and/or a buffer.

In some embodiments, the detergent and/or emulsifier is applied for a brief period (less than 15 minutes).

In some embodiments, the emulsifier is selected from a group comprising Lecithin, Polyoxyethylene Sorbitan Monolaurate (Polysorbate 20), Polyoxyethylene Sorbitan

Monooleate (Polysorbate 80), Polyoxyethylene Sorbitan Monopalmitate (Polysorbate 40), Polyoxyethylene Sorbitan Monostearate (Polysorbate 60), Polyoxyethylene Sorbitan Tristearate (Polysorbate 65), Ammonium Phosphatides, Sodium, Potassium and Calcium Salts of Fatty Acids, Magnesium Salts of Fatty Acids, Mono- and Diglycerides of Fatty Acids, Acetic Acid Esters of Mono- and Diglycerides of Fatty Acids, Lactic Acid Esters of Mono- and Diglycerides of Fatty Acids, Citric Acid Esters of Mono- and Diglycerides of Fatty Acids, Mono- and Diacetyl Tartaric Acid Esters of Mono- and Diglycerides of Fatty Acids, Mixed Acetic and Tartaric Acid Esters of Mono- and Diglycerides of Fatty Acids, Sucrose Esters of Fatty Acids, Sucroglycerides, Polyglycerol Esters of Fatty Acids, Polyglycerol Polyricinoleate, Propane-1,2-Diol Esters of Fatty Acids, Thermally Oxidised Soya Bean Oil Interacted with Mono- and Diglycerides of Fatty Acids, Sodium Stearoyl-2-Lactylate, Calcium Stearoyl-2-Lactylate, Sorbitan Monostearate, Sorbitan Tristearate, Sorbitan Monolaurate, Sorbitan Monooleate, Sorbitan Monopalmitate and combinations thereof.

In some embodiments, the detergent is selected from a group comprising 1-Heptanesulfonic Acid; N-Laurylsarcosine, Lauryl Sulfate, 1-Octane Sulfonic Acid and Taurocholic Acid, Benzalkonium Chloride, Cetylpyridinium, Methylbenzethonium Chloride, Decamethonium Bromide, Alkyl Betaines, Alkyl Amidoalkyl Betaines, N-Dodecyl-N,N-Dimethyl-3-Ammonio-1-Propanesulfonate, Phosphatidylcholine, N-Decyl A-D-Glucopyranoside, N-Decyl A-D-Maltopyranoside, N-Dodecyl B-D-Maltoside, N-Octyl B-D-Glucopyranoside, N-Tetradecyl B-D-Maltoside, Tritons (Triton X-100), Nonidet-P-40, Poloxamer 188, Sodium Lauryl Sulfate, Sodium Deoxycholate, Sodium Dodecyl Sulfate and combinations thereof.

In some embodiments, the remainder comprises a submucosal layer.

In some embodiments, digestion or dissociation is carried out enzymatically.

In some embodiments, the medium or solution to substantially kill, inactivate or remove pathogens and/or pathogenic and/or beneficial microbes comprises an aqueous solution of sodium hypochlorite (NaClO) or any solution(s) or agent(s) used for disinfection of skin or surfaces.

In some embodiments, the application of a medium or solution to substantially kill, inactivate or remove pathogens and/or pathogenic and/or beneficial microbes takes place before the application of the detergent and/or emulsifier, or takes place after the digestion or dissociation, or takes place after the removal of mucus.

In some embodiments, the tissue sample is minced before the digestion or dissociation step.

In some embodiments, the digestion or dissociation step and/or the isolation step is performed in low attachment plates.

In some embodiments, the isolation step is performed using culture selection with culture conditions that comprise a serum-free medium, optionally, Kubota's Medium.

In some embodiments, the isolation step is performed using culture selection with culture conditions that comprise a medium containing serum.

In some embodiments, the isolated cells are cultured under conditions that support or produce spheroids, one or more organoids, cell clusters, or cell aggregates.

In one aspect, the present disclosure relates to a spheroid, organoid, cell aggregate or cluster of cells produced by culturing the BGSCs, or the population of BGSCs in a low attachment plate.

In one aspect, the present disclosure relates to a spheroid, organoid, cell aggregate or cluster of cells produced by culturing the BGSCs, or the population of BGSCs in suspension or 3D culture conditions.

In one aspect, the present disclosure relates to a method of treating a subject diagnosed with a disease or condition involving or affecting the liver, pancreas, stomach, intestine, or other endodermal tissue comprising administering to a subject in need thereof an effective amount of BGSCs or a population of BGSCs.

In one aspect, the present disclosure relates to a method of treating a subject diagnosed with a disease or condition involving or affecting the liver, pancreas, stomach, intestine or other endodermal tissue comprising the administration of an effective amount of the BGSCs.

In one aspect, the present disclosure relates to a method of treating a subject diagnosed with a disease or condition involving or affecting the liver, pancreas, stomach, intestine or other endodermal tissue comprising the administration of an effective amount of the population of the BGSCs.

In one aspect, the present disclosure relates to a method of autologous cell or gene therapy comprising the administration of an effective number of BGSCs, or population of BGSCs.

In one aspect, the present disclosure relates to a method of allogeneic cell or gene therapy comprising the administration of an effective number of BGSCs, or population of BGSCs.

In one aspect, the present disclosure relates to a method of treating a subject diagnosed with a disease or condition involving or affecting the liver, pancreas, stomach, intestine, or other endodermal tissue comprising administering to a subject in need thereof an effective amount of BGSCs or a population of BGSCs, in which the cells are genetically engineered or modified cells.

In one aspect, the present disclosure relates to a method of treating a subject diagnosed with a disease or condition involving or affecting the liver, pancreas, stomach, intestine or other endodermal tissue comprising the administration of an effective amount of the BGSCs, in which the cells are genetically engineered or modified cells.

In one aspect, the present disclosure relates to a method of treating a subject diagnosed with a disease or condition involving or affecting the liver, pancreas, stomach, intestine or other endodermal tissue comprising the administration of an effective amount of the population of the BGSCs, in which the cells are genetically engineered or modified cells.

In one aspect, the present disclosure relates to a method of autologous cell or gene therapy comprising the administration of an effective number of BGSCs, or population of BGSCs, in which the cells are genetically engineered or modified cells.

In one aspect, the present disclosure relates to a method of allogeneic cell or gene therapy comprising the administration of an effective number of BGSCs, or population of BGSCs, in which the cells are genetically engineered or modified cells.

In one aspect, the present disclosure relates to a use of the cells of BGSCs for treatment of a disease or condition involving or affecting the liver, pancreas, stomach, intestine or other endodermal tissue for autologous or allogeneic cell or gene therapy for a human and/or an animal.

In one aspect, the present disclosure relates to a use of the cells of BGSCs for treatment of a disease or condition involving or affecting the liver, pancreas, stomach, intestine or other endodermal tissue for autologous or allogeneic cell or gene therapy for a human and/or an animal, in which the cells are genetically engineered or modified.

In one aspect, the present disclosure relates to a use of the population of the population of BGSCs for treatment of a disease or condition involving or affecting the liver, pancreas, stomach, intestine or other endodermal tissue, for autologous or allogeneic cell or gene therapy for a human and/or an animal.

In one aspect, the present disclosure relates to a use of the population of the population of BGSCs for treatment of a disease or condition involving or affecting the liver, pancreas, stomach, intestine or other endodermal tissue, for autologous or allogeneic cell or gene therapy for a human and/or an animal, in which the cells are genetically engineered or modified.

In some embodiments, the spheroid, organoid, cell aggregate or cluster of cells, further comprising culture conditions which are capable of differentiating BGSCs, or the population of BGSCs, into cells of later lineage stages, including mature cells.

In one aspect, the present disclosure relates to a method of isolating Brunner's Gland stem/progenitor cells (BGSCs), or the population of BGSCs, from a duodenum of a subject, a portion thereof, or a sample taken from same comprising:

digesting or dissociating a duodenum, a portion thereof, or a sample taken from same to provide a digest or dissociated cellular material;

obtaining from the digested or dissociated cellular material: (i) those cells that express, or a population of cells in which at least some, a substantial portion, or a majority of the cells expresses, one or more of Tra-1-60, Tra-1-81, OCT4, SOX2, NANOG, EpCAM, SOX9, CK7, Lgr5, NIS, CD44 and CK19; and/or (ii) those cells that express, or a population of cells in which at least some, a substantial portion, or a majority of the cells express, both SOX17 and PDX1.

In some embodiments, the duodenum, a portion thereof, a sample taken from same, the digest, the dissociated cellular material, or combinations thereof, are contacted with a medium or solution to substantially kill, inactivate or remove pathogens and/or pathogenic and/or beneficial microbes.

In one aspect, the present disclosure relates to a method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, comprising the following steps, which may occur in the following sequence or, in other embodiments, may occur in a different sequence:

contacting a mucosal layer of a tissue having a mucosal layer and a submucosal layer with a medium or solution having osmolality properties falling outside a physiological range under conditions that induce osmotic shock to the cells of the mucosal layer;

removing or dissolving at least a portion of the mucosal layer or the cells thereof by mechanical, surgical and/or chemical methods, leaving and/or exposing a remainder, which may include a submucosal layer;
contacting the remainder with a medium or solution to substantially kill, inactivate, or remove pathogens and/or pathogenic and/or beneficial microbes;
digesting or dissociating the remainder;
isolating one or more multipotent cells, or a population of cells in which at least some, or a substantial portion of, or a majority of the cells express one or more desired biomarkers.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, further comprising removal of surface mucus.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, in which the medium or solution having osmolality properties falling outside a physiological range comprises a hypotonic, hypoosmotic, hypertonic, or hyperosmotic solution.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the medium or solution having osmolality properties falling outside a physiological range comprises a glucose solution, a high salt solution, or distilled water.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the removal is carried out by chemical disruption, which comprises a use of an emulsifier and/or a detergent.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the detergent and/or emulsifier is in water, saline and/or a buffer.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the detergent and/or emulsifier is applied for a brief period (less than 15 minutes).

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the emulsifier is selected from a group comprising Lecithin, Polyoxyethylene Sorbitan Monolaurate (Polysorbate 20), Polyoxyethylene Sorbitan Monooleate (Polysorbate 80), Polyoxyethylene Sorbitan Monopalmitate (Polysorbate 40), Polyoxyethylene Sorbitan Mono stearate (Polysorbate 60), Polyoxyethylene Sorbitan Tristearate (Polysorbate 65), Ammonium Phosphatides, Sodium, Potassium and Calcium Salts of Fatty Acids, Magnesium Salts of Fatty Acids, Mono- and Diglycerides of Fatty Acids, Acetic Acid Esters of Mono- and Diglycerides of Fatty Acids, Lactic Acid Esters of Mono- and Diglycerides of Fatty Acids, Citric Acid Esters of Mono- and Diglycerides of Fatty Acids, Mono- and Diacetyl Tartaric Acid Esters of Mono- and Diglycerides of Fatty Acids, Mixed Acetic and Tartaric Acid Esters of Mono- and

Diglycerides of Fatty Acids, Sucrose Esters of Fatty Acids, Sucroglycerides, Polyglycerol Esters of Fatty Acids, Polyglycerol Polyricinoleate, Propane-1,2-Diol Esters of Fatty Acids, Thermally Oxidised Soya Bean Oil Interacted with Mono- and Diglycerides of Fatty Acids, Sodium Stearoyl-2-Lactylate, Calcium Stearoyl-2-Lactylate, Sorbitan Monostearate, Sorbitan Tristearate, Sorbitan Monolaurate, Sorbitan Monooleate, and Sorbitan Monopalmitate.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the detergent is selected from a group comprising 1-Heptanesulfonic Acid; N-Laurylsarcosine, Lauryl Sulfate, 1-Octane Sulfonic Acid and Taurocholic Acid, Benzalkonium Chloride, Cetylpyridinium, Methylbenzethonium Chloride, Decamethonium Bromide, Alkyl Betaines, Alkyl Amidoalkyl Betaines, N-Dodecyl-N,N-Dimethyl-3-Ammonio-1-Propanesulfonate, Phosphatidylcholine, N-Decyl A-D-Glucopyranoside, N-Decyl A-D-Maltopyranoside, N-Dodecyl B-D-Maltoside, N-Octyl B-D-Glucopyranoside, N-Tetradecyl B-D-Maltoside, Tritons (Triton X-100), Nonidet-P-40, Poloxamer 188, Sodium Lauryl Sulfate, Sodium Deoxycholate, and Sodium Dodecyl Sulfate.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the medium or solution to substantially kill, inactivate or remove pathogens and/or pathogenic and/or beneficial microbes comprises an aqueous solution of sodium hypochlorite (NaClO) or any solution(s) or agent(s) used for disinfection of skin or surfaces.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the application of a medium or solution to substantially kill, inactivate or remove pathogens and/or pathogenic and/or beneficial microbes takes place before the application of the detergent and/or emulsifier, or takes place after the digestion or dissociation, or takes place after the removal of mucus.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the remainder comprises a submucosal layer.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the digestion or dissociation is carried out enzymatically.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the tissue sample is minced before the digestion or dissociation step.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the digestion or dissociation breaks down submucosal tissue into a cell suspension, mixture of cells, clusters, clumps or aggregates, and/or tissue fragments.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the isolation step is performed using culture selection with culture conditions comprising a serum-free medium, optionally, Kubota's Medium.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the isolation step is performed using culture selection with culture conditions comprising a medium containing serum.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the isolation step is performed in low attachment plates.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, the isolated cells or cell population are cultured under conditions that support or produce spheroids, one or more organoids, cell clusters, or cell aggregates

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, further comprises one or more wash steps using a physiologically acceptable medium.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, in which the tissue is an endodermal tissue.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, in which the tissue is selected from a group comprising trachea, main bronchus, esophagus, stomach, duodenum, small intestine, large intestine and rectum.

In some embodiments, the method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, in which the tissue is selected from a group comprising liver, pancreas, gall bladder and biliary tree ducts, wherein the biliary tree ducts comprise common ducts and cystic ducts.

### BRIEF DESCRIPTION OF THE DRAWINGS

FIG. 1 depicts A) Human duodenum stained with Periodic Acid-Schiff (PAS). Duodenal mucosa is elevated into intestinal villi and folded in intestinal crypts (arrowheads). The submucosa (SM) is replete with PAS⁺ glandular elements (Brunner's Glands: BGs, dotted line). BGs are in anatomical continuity with intestinal crypts through the muscolaris mucosae (MM). Few BG acini are located inside the lamina propria of the mucosa and are in continuity with intestinal crypts (arrows in right image). B) Immunohistochemistry for Cytokeratin 7 (CK7) in human duodenum. CK7 is expressed specifically by BGs but not by intestinal crypts. C) Immunohistochemistry for SOX9 in human duodenum. Both intestinal crypts and BGs contain cells expressing SOX9 (arrows). D) Immunofluorescence for SOX9 (red) and CK7 (green); nuclei are displayed in blue. In BGs, SOX9 is co-expressed with CK7 in the same cells (arrows).

FIG. 2 depicts A) Immunohistochemistry for Proliferating Cell Nuclear Antigen (PCNA), CD44, Epithelial Cell Adhesion Molecule (EpCAM), Leucine-Rich Repeat Containing G Protein-Coupled Receptor 5 (Lgr5), Tra-1-60, and Tra-1-81 in human duodenum. PCNA⁺, CD44⁺, EpCAM⁺, and Lgr5⁺ cells are located both in intestinal crypts (arrows) and in Brunner's glands (arrows). Tra-1-60⁺ and Tra-1-81⁺ cells are located in Brunner's glands (arrows) but not in intestinal crypts. MM=muscolaris mucosae. B) Immunohistochemistry and immunofluorescence for transcriptional factors associated with pluripotency. Oct4A and SOX2 are positive in cells within Brunner's Glands and are co-expressed in Tra-1-60⁺ cells (arrows).

FIG. 3 depicts A) Hematoxylin and Eosin (H&E) stained sections of the human duodenum before and after the chemical and mechanical removal of the mucosa. Almost all epithelial cells within the surface epithelium (villi) and intestinal crypts (arrowheads) were removed with the exception of rare intestinal crypts (dotted circle in the right image). Brunner's glands in the submucosa are preserved (asterisks). B) Immunohistochemistry for Cytokeratin 7 (CK7) in human duodenum after the mechanical and chemical removal of the mucosa. CK7⁺ cells in Brunner's glands are preserved. C) Immunohistochemistry for Tra-1-60 in human duodenum after the mechanical and chemical removal of the mucosa. Tra-1-60⁺ cells in Brunner's glands are preserved. MM=muscolaris mucosae.

FIG. 4 depicts A-B) Flow cytometry for Epithelial Cell Adhesion Molecule (EpCAM), Leucine-Rich Repeat Containing G Protein-Coupled Receptor 5 (Lgr5), and Tra-1-60 in cells isolated from duodenal submucosa. EpCAM and Tra-1-60 immunosorting procedures resulted in the partial enrichment of the EpCAM (A) and Tra-1-60⁺ (B) populations, respectively. C-D) Culture selection strategies further selected Tra-1-60⁺ cells. In C, cells were plated at a clonal density on plastic in Kubota's Medium. Cells started to proliferate after a 1-2 days lag period and formed small clusters of 10-15 cells after 6-8 days. After 14 days, large colonies were observed. Each colony was formed by Tra-1-60⁺ small, densely packed, and uniform cells. In D, cells were cultured in conditions tailored for organoid formation; single Brunner's Gland Stem Cells (BGSCs) started to self-organize into spherical structure that further expanded in size and number. Organoid formation determined the enrichment for Tra-1-60⁺ cells that represent the predominant phenotype forming the organoids. Ph-C: phase contrast. Scale bars=200 μm.

FIG. 5 depicts A-B) Phase contrast (Ph-C), Hematoxylin & Eosin (H&E) and Periodic Acid-Schiff (PAS) stains, immunohistochemistry and immunofluorescence for endoderm and pluripotency markers. In panel A, Brunner's Gland Stem Cells (BGSCs) were cultured under self-replication conditions (i.e. serum-free Kubota's Medium). In panel B, BGSCs were cultured under conditions tailored for organoid formation. In both conditions, cultured cells showed a typical phenotype which is comparable with the one observed in situ by cells forming Brunner's Glands. Nuclei are displayed in blue. C-D) RT-PCR analysis confirmed the expression of endoderm (C) and pluripotency (D) genes. Biliary Tree Stem/progenitor cells (BTSCs) and the Ntera cell line were used as positive controls for endoderm and pluripotency genes, respectively. *, p<0.05 versus the other group. GOI=Gene of Interest.

FIG. 6 depicts A) In vitro hepatocyte differentiation. Phase contrast (Ph-C), Periodic Acid—Schiff (PAS) stain and immunofluorescence for albumin in human Brunner's Gland Stem/progenitor Cells (hBGSCs) cultured in a hormonally defined medium for hepatocyte differentiation (HDM-H). After 14 days in HDM-H, the morphology of most cells changed noticeably to polygonal-shaped cells. These cells aggregated to form multicellular cords, were PAS positive (glycogen storage) and expressed albumin. Nuclei are displayed in blue. Real time PCR for hepatocyte markers in cells cultured in HDM-Liver for 14 days. Hepatocyte-specific genes, including albumin (ALB), transferrin (TF), and Cytochrome P450 3A4 (CYP3A4), were increased when compared with cells under self-replication conditions (i.e. serum-free Kubota's Medium: KM). Primary human hepatocytes (hHeps) are used as positive control. *, p<0.05 versus other groups. GOI=Gene of Interest. B) In vitro endocrine pancreatic differentiation. Ph-C, Hematoxylin & Eosin (H&E) stain and immunofluorescence for neurogenin 3 (NGN3) and insulin in human Brunner's Gland Stem/progenitor Cells (hBGSCs) cultured in a hormonally defined medium for pancreatic islet cell differentiation (HDM-P). After 14 days in HDM-P, islet-like structures appeared in cultures; these aggregates were NGN3 and insulin positive. Nuclei are displayed in blue. Real time PCR for pancreatic endocrine markers in cells cultured in HDM-P for 14 days. PDX1, insulin and glucagon were highly increased when compared with cells in self-replication conditions (i.e. Kubota's Medium: KM). Normal pancreatic islet cells were used as positive controls. *, p<0.05 versus hBGSCs in KM. GOI=Gene of Interest.

FIG. 7 depicts in vivo transplantation of human Brunner's Glands Stem Cells (hBGSCs) into the livers of SCID mice by intrasplenic injection. A) After 30 days, cells expressing human mitochondrial antigen (hMito) were present in murine livers and mostly located around portal triad spaces (arrows). No positive cells were present in mice injected with saline (Veh). B) hMito positive cells accounted for nearly 5% of the hepatocytes in the murine livers. C) The expression of human albumin (hALB) gene was detected by RT-qPCR in liver of mice injected with hBGSC but not in mice injected with Veh (ND: not detected). Data expressed as mean±SD for three experiments. D-E) Double immunofluorescence confirmed the expression of markers of mature human hepatocyte such as human albumin (hAlb), Hep-Parl, and hMito in the same cells. Nuclei (Nu) were displayed in blue.

FIG. 8 depicts selective solubilization of the duodenum mucosa. A) Lavage method; B) clamping of duodenum extremities; C) tissue slitting and mucus removing; D) mechanical filtering by using colander.

FIG. 9 depicts immunohistochemistry for Cytokeratin 7 (CK7) in human duodenum. Duodenal wall is composed of Mucosa, Submucosa and Muscolar layers. Brunner's Glands (BG) are located within the submucosal layer. CK7 is expressed specifically by BGs but not by cells in intestinal crypts. Areas in the boxes are magnified in images on the right.

FIG. 10 depicts A) Immunofluorescence for SOX9 (red) and Lgr5 (green) in human duodenum. Nuclei are displayed in blue. In Brunner's glands (comprised into the dotted line), Lgr5 co-localized with SOX9 (arrows), and its expression was greater in acini located inside the muscolaris mucosae and in continuity with intestinal crypts (arrowheads) than in acini deeper located within the submucosal layer. MM=muscolaris mucosae. B-D) Immunohistochemistry for Sodium/Iodide Symporter (NIS). NIS is expressed at the bottom of intestinal crypts (arrowheads in panels A and C) and in Brunner's glands (arrows in panels A and B).

FIG. 11 depicts unsuccessful protocols and procedures for the removal of mucosa epithelial cells combined with the preservation of submucosa viability. Procedure #1: surgical dissection, #2 mucosectomy by previous injection of normal saline under the mucosa, #3 scraping the mucosa. These strategies resulted in partial removal of mucosa layer and the preservation of intestinal villi (arrowheads) and crypts (arrows) as showed in Hematoxylin & Eosin (H&E) and Periodic Acid-Schiff (PAS) stains.

FIG. 12 depicts real time PCR in human Brunner's Gland Stem/progenitor Cells (hBGSCs) cultured under a hormonally defined medium for pancreatic islet cell differentiation (HDM-P) for 7 days. PDX1 and glucagon genes but not insulin gene are highly increased after 7 days when compared with cells under self-replication conditions (i.e. Kubota's Medium: KM). Normal pancreatic islet cells were used as positive control. *, p<0.05 versus hBGSCs in KM. GOI=Gene of Interest.

FIG. 13 depicts that duodenal submucosal glands in mice represent a distinct compartment with respect to intestinal crypts, and shows proliferative features. A) depicts Hematoxylin and Eosin (H&E) staining, and immunofluorescence staining for Cytokeratin 19 (Ck19), SOX9 and Proliferating Cell Nuclear Antigen (PCNA) in murine (m) duodenum. Dotted line individuates the interface between intestinal crypts and submucosal glands (SGs: asterisk). SGs in duodenum are distinguishable because they have clearer cytoplasm compared to crypts and because of their mucous content. In rodent duodenum, intestinal crypts and villi are Ck19 positive while SGs (white asterisk) are almost negative. SOX9+ cells are mainly located within SGs (green cells) and PCNA+ cells are mainly located in intestinal crypts (red cells). Nuclei are displayed in blue. Scale bars=200 μm (H&E and Ck19) or 100 μm (SOX9/PCNA). B) depicts Hematoxylin and Eosin staining and immunofluorescence staining for Ck19, SOX9 and PCNA in murine (m) jejunum. In rodent jejunum, intestinal crypts and villi are Ck19 positive. Distinctions with respect to duodenum are that SOX9+ cells are located in crypts and co-express PCNA (yellow arrows). Nuclei are displayed in blue. Scale bars=200 μm (H&E and Ck19) or 100 μm (SOX9/PCNA). C) depicts immunofluorescence for Ck19 and TdTomato (Td-Tom) in Krt19CreTdTomatoLSL mice 14 days after tamoxifen injection. In murine (m) jejunum, most intestinal crypts are Td-Tom+(red arrows) with negative crypts (green arrow) located closeto positive ones. Villi located above Td-Tom+ crypts are completely td-Tom positive. In murine duodenum, SGs are mostly td-Tom- and Ck19-, thus excluding their origin from the td-Tom+ crypts (red arrows). White asterisks indicate SGs. Dotted lines individuate the interface between intestinal crypts and SGs. Nuclei are displayed in blue. Scale bars=200 D) In murine duodenum, PCNA+505 and SOX9⁺ cells are always Td-Tom negative (red arrows). Yellow and green arrows point Td-Tom+ intestinal crypts that in duodenum are PCNA positive and SOX9 negative. Nuclei are displayed in blue. Dotted lines individuate the interface between intestinal crypts and SGs. Images in FIG. 13 were representative of n=5 animals.

FIG. 14 depicts Tra-1-60⁺ cells isolated from duodenal submucosa can be restricted to endocrine pancreas in vitro and show in vivo potency to differentiate into insulin⁺ cells. A-C) In vitro endocrine pancreatic differentiation. Panel A) shows phase contrast (Ph-C) and Hematoxylin & Eosin (H&E) stain of cells isolated from human duodenal submucosal glands and cultured in a defined medium for pancreatic differentiation (PM) or in self-replication conditions (Kubota's Medium: KM). In PM, islet-like structures appear after 7 days (PM7) and increase in number after 14 days (PM14); *p<0.001 versus other groups. Scale bars=100 μm. n=5 biological replicates. B) Real time (RT)-PCR shows increased PDX1 gene expression in PM7-14; 583 nuclear Pdxl expression is confirmed by immunofluorescence. n=4 biological replicates. C) depicts that insulin (INS) and glucagon (GLU) gene expression increase after in PM14 (n=4 biological replicates). Human pancreatic islet cells (ISL) are used as reference (n=3 biological replicates). In 14-day PM, islet-like structures show insulin and glucagon expression by immunofluorescence. Scale bar=100 μm. D-G) depicts that experimentally-induced diabetes in mice can trigger proliferation and pancreatic traits in dSGs in vivo (n=5 animals for each group). D) depicts that streptozotocin (STZ) treated mice have increased extent of dSG area fraction (asterisks) compared to controls (CTR). Dotted lines individuate intestinal crypts from dSGs. Scale bars=200 μm. E) depicts that dSGs in STZ mice have increased expression of Proliferating Cell Nuclear Antigen (PCNA), Pdx-1, Neurogenin3 (Ngn3), and Insulin compared to controls as determined by immunofluorescence (IF). Scale bars=100 F) depicts a heat map of the IF-score. G) depicts that specimens from rodent duodenum have a slightly increased expression of NGN3 and INS genes in STZ mice compared to controls as determined by RT-PCR analysis. Pancreatic tissues of the same mice are used as reference. H) depicts studies of insulin expression in human duodena obtained from patients affected by Type 2 Diabetes (T2D). In these organs (n=5 duodena), rare insulin⁺ cells are present within SGs (arrows). Pancreatic tissue is shown on the right. Scale bars=50 μm. In immunofluorescence, nuclei are displayed in blue. For A-D and G, error bars indicate mean±s.d. For A-D and G, p-values were determined by two-tailed t-test.

FIG. 15 depicts obtaining self-replicating Brunner's Gland cells obtained by endoscopic biopsis of human duodenal bulb. A) shows collection of submucosal layer with Brunner's Glands (asterisk) by biopsy (left panel). SOX9 expressing cells in the Brunner's Gland are indicated with arrows (right panel). B) shows in vitro cell colonies (dotted lines) obtained from culturing Brunner's Gland Cells isolated from fetal duodenum in self-replicating conditions.

### DETAILED DESCRIPTION

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but do not exclude others. As used herein, the transitional phrase “consisting essentially of”(and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace or minor elements of other ingredients and substantial method steps for administering the compositions disclosed herein. Aspects defined by each of these transition terms are within the scope of the present disclosure.

The terms “equivalent” or “biological equivalent” are used interchangeably when referring to a particular molecule, biological, or cellular material and intend those having minimal homology while still maintaining desired structure or functionality.

As used herein, the term spheroid is used when referring to an aggregate of substantially or primarily the same type of cells that have organized into a three dimensional (3D) structure enabling the cells to interact in a suspension culture environment.

As used herein, the term organoid is used when referring to an aggregate of one or more types of cells that have organized into a three dimensional (3D) structure enabling the cells to interact in a suspension culture environment. In some cases, an organoid may mimic aspects of the structure and function of a [human or animal] organ or tissue.

As used herein, the term microbe refers to a microorganism which may or may not be pathogenic or causing a disease, or may or may not be beneficial, that may reside inside or outside a tissue being processed to obtain desired cells or cell population.

As used herein, the term “expression” refers to the process by which DNA or polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined, for example by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes certain effect.

The term “gene” or “genetic” as used herein is meant to broadly include any nucleic acid sequence, which may or may not be transcribed into an RNA molecule, whether the DNA or RNA is coding (e.g., mRNA) or non-coding (e.g., ncRNA). The terms “nucleic acid,” “polynucleotide,” and “oligonucleotide” are used interchangeably and refer to a polymeric form of nucleotides of any length, either deoxyribonucleotides or ribonucleotides or analogs thereof. Polynucleotides can have any three dimensional structure and may perform any function, known or unknown. The following are non-limiting examples of polynucleotides: a gene or gene fragment (for example, a probe, primer, EST or SAGE tag), exons, introns, messenger RNA (mRNA), microRNA, transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers.

The term “genetically engineered or modified” as used herein is meant to broadly include any form of modification of a cell or its genetic material, including but not limited to deletion, addition or modulation of genes or genetic material, recombinant-DNA technology, genetic modifications through viral vectors or electroporation, gene targeting or editing through CRISPER (Clustered Regularly Interspaced Short Palindromic Repeat) or otherwise, deletion or addition of a DNA fragment, correction of a genetic mutation, and so on.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Unless otherwise specified or required, any aspect of this technology that is a polynucleotide encompasses both the double stranded form and each of two complementary single stranded forms known or predicted to make up the double stranded form.

The term “protein,” “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “subject” and “patient” are used interchangeably and are intended to mean any human or animal. In some embodiments, the subject may be a mammal. In further embodiments, the subject may be a human or non-human animal (e.g. a mouse or rat).

The term “tissue” is used herein to refer to tissue of a living or deceased organism or any tissue derived from or designed to mimic some aspects of a living or deceased organism. The tissue may be healthy, diseased, and/or have genetic mutations or modifications. The term “natural tissue” or “biological tissue” and variations thereof as used herein refer to the biological tissue as it exists in its natural state or in a state unmodified from when it was derived from an organism. A “micro-organ” refers to a segment of “bioengineered tissue” that is modeled on or mimics “natural tissue.”

The biological tissue may include any single tissue (e.g., a collection of cells that may be interconnected) or a group of tissues making up an organ or part or region of the body of an organism. The tissue may comprise homogeneous or heterogeneous cellular material, or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

The term “isolated” as used herein refers to molecules or biologicals or cellular materials which are substantially free from other materials. The term “isolated” is also used to describe materials that have been removed from their natural environment (e.g., from in vivo to ex vivo or in vitro). The term “sterile” as used herein refers to a material that is free from bacteria or other living microorganisms (i.e., aseptic, sterilized, germ-free, antiseptic, disinfected, or the like).

Isolated Brunner's Gland Stem/Progenitor Cells and Cell Culture Medium

As used herein, the term “cell” refers to a eukaryotic cell. In various embodiments, this cell is of human or animal origin and can be a stem or progenitor cell or a somatic cell. The term “population of cells” refers to a group of one or more cells of the same or different cell type in which at least some, or a substantial portion or a majority of the cells have the same or different origin and/or lineage stage. In some embodiments, this population of cells may be derived from a cell line; in some embodiments, this population of cells may be derived from a an an organ or tissue, a portion thereof or a sample of same.

The term “stem cell” refers to cell populations that can self-replicate (produce daughter cells identical to the parent cell) and that are multipotent, i.e. can give rise to more than one type of adult cell. The term “progenitor cell” or “precursor” as used herein, is also multipotent, although the scope or extent of the multipotency of a progenitor cell or precursor may be more limited than the multipotency of a stem cell. The term “progenitor cell” or “precursor” is also broadly defined to encompass progeny of stem cells and their descendants. Progenitors are cell populations that can be multipotent, bipotent, or unipotent, but may have more limited ability to self-replicate than stem cells. Committed progenitors are ones that can differentiate into a particular lineage. Non-limiting examples of stem cells include but are not limited to embryonic stem (ES) cells, induced pluripotent stem (iPS) cells, germ layer stem cells, determined stem cells, adult stem cells, perinatal stem cells, amniotic fluid-derived stem cells, mesenchymal stem cells (MSCs), and angioblasts. Intermediates between stem cells and committed progenitors include cell populations such as hepatoblasts and pancreatic ductal progenitors and other forms of transit amplifying cells that may be multipotent but may have limited self-replicative ability.

The cells of interest in the present disclosure are referred to herein as Brunner's Gland stem/progenitor cells (BGSCs) and may be derived from the duodenum of a human or animal (but nowhere else in the intestine). These BGSCs are distinguishable from the intestinal stem cells based on at least the following features:

| | |
|---|---|
| BGSCs | Intestinal Stem Cells* |

PHENOTYPIC TRAITS (MARKERS)

| | | |
|---|---|---|
| Tra-1-60 | + | − |
| Tra-1-81 | + | − |
| OCT4A | + | − |
| Cytokeratin 7 | + | − |
| Cytokeratin 19 | + | + |
| EpCAM | + | + |
| SOX9 | + | + |
| CD44 | + | + |
| NIS | + | + |
| Lgr5 | +/− | + |

FUNCTIONAL PROPERTIES
(ORGANOID FORMATION and
DIFFERENTIATION CAPABILITIES)

| | | |
|---|---|---|
| Organoid | + | +¹ |
| Formation | | |
| Endocrine | + | −² |
| Pancreas lineages | | |
| Liver lineages | + | −² |

Applicants were the first to recognize duodenum and its Brunner's glands as important stem/progenitor cell niches that are part of a network of stem/progenitor niches in glands located in the submucosa of the duodenum, giving rise to liver, pancreas and other endodermal cells and tissues, and persisting into adulthood. The BGSCs are a small subpopulation (e.g. ˜5%) of the cells within the Brunner's Glands (also known as duodendal submucosal glands) and are recognizable as those expressing pluripotency genes such as TRA-1-60, Tra-1-81, OCT4, SOX2, and NANOG. The BGSCs are relevant to liver, biliary tree, pancreas, intestine and other endodermal tissues having biomarkers that may include Lgr5, NIS, CD44, CK19, SOX9, and EpCAM, and/or may include both SOX17 and PDX1.

The BGSCs are distinct from intestinal stem cells as described above: the BGSCs express Tra-1-60, Tra-1-81, OCT4 and CK7 while intestinal stem cells do not. The BGSCs are also distinct from hepatic stem cells and pancreatic stem cells in that the BGSCs express both SOX17 and PDX1, whereas the hepatic stem cell ones express SOX17 but not PDX1, and the pancreatic stem cells express PDX1 but not SOX17.

Accordingly, in one aspect, this disclosure relates to a BGSC isolated from a duodenum that expresses one or more of the markers selected from the group consisting of Tra-1-60, Tra-1-81, OCT4, SOX2, NANOG, EpCAM, SOX9 and CK7, and which is further characterized as capable of proliferation, with limited or minimal differentiation, under culture conditions that support self-renewal. In another aspect the BGSC isolated from a duodenum expresses one or more markers selected from the group consisting of Lgr5, NIS, CD44 and CK19, and the BGSC is further characterized as capable of proliferation, with limited or minimal differentiation, under culture conditions that support self-renewal. In another aspect, the BGSC isolated from a duodenumexpresses both SOX17 and PDX1, and the BGSC is further characterized as capable of proliferation, with limited or minimal differentiation, under culture conditions that support self-renewal. In some embodiments, the isolated BGSC may express one or more markers from the group consisting of Tra 1-60, Tra-1-81, OCT4, SOX2, NANOG, EpCAM, SOX9, CK7, Lgr5, NIS, CD44, CK19, and both SOX17 and PDX1. In some embodiments, the isolated BGSC is substantially free of pathogens and/or free of pathogenic and/or beneficial microbes.

In some embodiments, the isolated BGSC can be proliferated, with limited or minimal differentiation, for at least one month. In some embodiments, the isolated BGSC can be proliferated, with limited or minimal differentiation, for at least two months. In some embodiments, the isolated BGSC can be proliferated, with limited or minimal differentiation, for at least six months or for at least twelve months.

In some embodiments, the culture conditions that support self-renewal of the BGSC comprise a serum-free medium. In some embodiments, the serum-free medium comprises Kubota's medium.

The term “culture” or “cell culture” means the maintenance of cells in an in vitro environment. A “cell culture system” is used herein to refer to culture conditions in which a population of cells may be grown ex vivo (outside of the body). Cultures may be comprised of a single type of cell or a mixture of different cell types.

Cell cultures include ones that are monolayers in which cells are plated onto a surface with or without a coating, such as a coating of extracellular matrix components, and in a nutrient medium (minerals, vitamins, amino acids, lipids) supplemented with either a biological fluid (e.g. serum or lymph) and/or with a defined mixture of hormones, growth factors and cytokines (a hormonally defined medium or HDM). The HDM are defined empirically for their usefulness with a particular type of cell or a population of cells, at a particular maturational lineage stage.

Cell cultures can also be floating clusters or aggregates of cells plated onto low attachment dishes and/or can be in suspension culture. The media supportive of floating clusters or aggregates, and/or of suspension culture, can be same media used for the monolayer culture. The media can be serum-free or can contain serum. Serum-free media lacks attachment proteins (e.g., fibronectins) that can cause floating aggregates to generate monolayers. If the floating aggregates are comprised substantially or primarily of one cell type, they are referred to as spheroids. If they are comprised of multiple cell types [e.g., epithelia and a mesenchymal cell partner(s)], they are referred to as organoids. b Cell clusters or aggregates, spheroids and organoids floating in suspension cultures are considered to be in a 3D microenvironment, and the cells are able to interact in three dimensions.

“Culture medium” is used herein to refer to a nutrient solution for the culturing, growth, or proliferation of cells. Culture medium may be characterized by functional properties such as, but not limited to, the ability to maintain cells in a particular state (e.g. a pluripotent state, a quiescent state, etc.), to facilitate maturation of cells, and in some instances to promote the differentiation of multipotent cells into cells of a particular lineage.

Non-limiting examples of culture media are serum supplemented media (SSM), being any basal medium supplemented with serum (derived from animals routinely slaughtered for commercial and agricultural products) at levels that are typically ˜10%.

The term “isolated” as used herein refers to molecules or biologicals or cellular materials which are substantially free from other materials (except culture medium and/or extracellular matrix, and their respective components). The term “isolated” is also used to describe materials that have been removed from their natural environment (e.g., from in vivo to ex vivo or in vitro).

The term “sterile, sanitized or disinfected” as used herein refers to a material that is free from pathogens and/or pathogenic and/or beneficial microbes (i.e., aseptic, sterilized, germ-free, antiseptic, disinfected, or the like).

Isolated Population of BGSCs

In another aspect, this disclosure relates to a population of stem/progenitor cells isolated from duodenum in which at least some, or a substantial portion of, or a majority of the cells express one or more of the markers selected from the group consisting of Tra-1-60, Tra-1-81, OCT4, SOX2, NANOG, EpCAM, SOX9 and CK7 and which is further characterized as capable of proliferation, with limited or minimal differentiation, under culture conditions that support self-renewal. In another aspect, this disclosure relates to a population of stem/progenitor cells isolated from duodenum in which at least some, or a substantial portion of, or a majority of the cells express one or more of the markers selected from the group consisting of Lgr5, NIS, CD44 and CK19. In another aspect, this disclosure relates to a population of stem/progenitor cells isolated from duodenum in which at least some, or a substantial portion of, or a majority of the cells express both SOX17 and PDX1. In some embodiments, at least some, or a substantial portion of, or a majority of the cells in the stem/progenitor population isolated from duodenum express one or more markers from the group consisting of Tra 1-60, Tra-1-81, OCT4, SOX2, NANOG, EpCAM, SOX9, CK7, Lgr5, NIS, CD44, CK19, and both SOX17 and PDX1.

In some embodiments, the isolated population of stem/progenitors described in the preceding paragraph is substantially free of pathogens and/or free of pathogenic and/or beneficial microbes

In some embodiments, the present disclosure relates to a composition comprising an isolated BGSC population, expressing one or more markers selected from the group consisting of Tra-1-60, Tra-1-81, OCT4, SOX2, NANOG EpCAM, SOX9, CK7, Lgr5, NIS, CD44, CK19, CD44, CK19, and both, SOX17 and PDX1, and in some embodiments this BGSC population has been sterilized, sanitized or disinfected.

In another aspect, this disclosure relates to an organoid produced by culturing an isolated BGSC population in which at least some, or a substantial portion of, or a majority of the cells express one or more markers selected from the group consisting of Tra 1-60, Tra-1-81, OCT4, SOX2, NANOG, EpCAM, SOX9 and CK7, Lgr5, NIS, CD44 and CK19, and both SOX17 and PDX1, and which is further characterized as capable of proliferation, with limited or minimal differentiation, under culture conditions that support self-renewal, optionally on a low attachment plate or in suspension. In some embodiments, the organoid further comprises a culture medium, wherein the culture medium is capable of differentiating BGSCs into later lineage stage cells, including mature cells.

In some embodiments, the culture conditions that support self-renewal comprise a serum-free medium. In some embodiments, the serum-free medium comprises Kubota's medium.

In some embodiments, the serum-free culture conditions are hormone defined medium (HDM) designed for particular maturational lineage stages of cells, whether epithelia or mesenchymal cells. In addition to SSM, there are serum-free, HDM media designed for particular maturational lineage stages of cells, whether epithelia or mesenchymal cells. An example of this is Kubota's Medium, a serum-free medium designed for endodermal stem/progenitors and comprised of a basal medium (nutrient medium containing minerals, amino acids, sugars, salts, vitamins, lipids) with no copper and low calcium and supplemented with insulin, transferrin/Fe, and various lipids, but no cytokines or growth factors. This medium can support endodermal stem/progenitor cells from liver, pancreas, lung, and intestine.

“Kubota's Medium” as used herein refers to any medium containing no copper, but containing calcium (<0.5 mM), selenium, zinc, insulin, transferrin/Fe, a mix of free fatty acids bound to purified albumin and, optionally, also high density lipoprotein (HDL). In some embodiments, Kubota's Medium comprises any medium (e.g., RPMI 1640 or DMEM-F12) with no copper, low calcium (e.g., 0.3 mM), ˜10-9 M selenium, ˜0.1% bovine serum albumin or human serum albumin (highly purified and fatty acid free), ˜4.5 mM nicotinamide, ˜0.1 nM zinc sulfate heptahydrate, ˜10-8 M hydrocortisone (optional component used for hepatic but not pancreatic precursors), ˜5 μg/ml transferrin/Fe, ˜5 μg/ml insulin, ˜10 μg/ml high density lipoprotein, and a mixture of purified free fatty acids that are added after binding them to purified serum albumin. The free fatty acid mixture consists of ˜100 mM each of palmitic acid, palmitoleic acid, oleic acid, linoleic acid, linolenic acid, and stearic acid. Non-limiting, exemplary methods for the preparation of this media have been published elsewhere, e.g., Kubota H, Reid L M, Proc. Nat. Acad. Scien. (USA) 2000; 97:12132-12137, the disclosure of which is incorporated herein.

There are other serum-free HDM that can be designed to drive the stem/progenitors to specific adult fates such as hepatocytes (HDM-H) or cholangiocytes (HDM-C). Some of these HDM are further defined herein below. In some embodiments the medium may be a “seeding medium” used to present or introduce cells into a given environment. In other embodiments, the medium may be a “differentiation medium” used to facilitate the differentiation of cells.

Such media are comprised of a “basal medium”, a mixture of nutrients, minerals, amino acids, sugars, lipids, and trace elements (examples include Dulbecco's Modified Eagle's Medium or DME and Ham's F10 or F12 and RPMI 1640, a defined basal medium established at the Roswell Park Memorial Institute). These basal media can be supplemented either with serum (serum supplemented media or SSM) or with a defined mix of purified hormones, growth factors and nutrients, a hormonally defined medium (HDM), and used for maintenance of cells ex vivo. As used herein, “HDM-H” is an HDM used for organoids or for monolayer cultures plated onto substrata of type IV collagen and laminin to drive the differentiation of endodermal stem/progenitors to mature hepatocytes. HDM-C is an HDM used for organoids or used for monolayers plated onto substrata of type I collagen and fibronectin to drive the cells to mature cholangiocytes.

Particular Hormonally Defined Media (HDM) compositions are described in greater detail below:

Modified KM (MKM): All three of the HDM below made use of KM supplemented further with calcium to achieve a 0.6 mM concentration, 10⁻¹² M copper, and 20 ng/ml of FGF.

Hepatocyte differentiation (HDM-L): was prepared supplementing MKM with 7 μg/L glucagon, 2 g/L galactose, 1 nM triiodothyroxine 3 (T3), 10 ng/ml Oncostatin M (OSM); 10 ng/ml epidermal growth factor (EGF), 20 ng/ml hepatocyte growth factor (HGF), and 1 μm dexamethasone.

Cholangiocyte differentiation (HDM-C): was prepared by supplementing the MKM with 20 ng/ml vascular endothelial cell growth factor (VEGF) 165 and 10 ng/ml HGF.

Defined medium for pancreatic differentiation (PM or HDM-P): was prepared using MKM without hydrocortisone and further supplemented with 2% B27, 0.1 mM ascorbic acid, 0.25 cyclopamine, 1 μM retinoic acid, the bFGF was used for the first 4 days and replaced with 50 ng/ml exendin-4 and 20 ng/ml of HGF for the remainder of the time.

Basal media are buffers used for cell culture and are comprised of amino acids, sugars, lipids, vitamins, minerals, salts, trace elements, and various nutrients in compositions that mimic the chemical constituents of interstitial fluid around cells. In addition, cell culture media are usually comprised of basal media supplemented with a small percentage (typically 2-10%) serum to provide requisite signaling molecules (hormones, growth factors) needed to drive a biological process (e.g., proliferation, differentiation). Although the serum can be autologous to the cell types used in cultures, it is most commonly serum from animals routinely slaughtered for agricultural or food purposes such as serum from cows, sheep, goats, horses, etc. Serum is also used to inactivate enzymes that are part of tissue dissociation processes.

Methods of Isolating Multipotent Cells from a Tissue Having a Mucosal Layer and A Submucosal Layer

In another aspect, this disclosure relates to a method of isolating one or more multipotent stem/progenitor cells expressing one or more desired biomarkers, or a population in which at least some, or a substantial portion of, or a majority of the cells express the desired biomarker(s), from a tissue having a mucosal layer and a submucosal layer comprising:
contacting a mucosal layer of a duodenum, which is substantially free of intestinal mucus, with a medium or solution having osmolality properties falling outside a physiological range under conditions that induce osmotic shock to the cells of the mucosal layer;
removing or dissolving at least a portion of the mucosal layer or the cells thereof by mechanical, surgical and/or chemical methods, leaving and/or exposing a remainder which may include a submucosal layer;
digesting or dissociating the remainder; and
isolating one or more BGSC or the population of cells from the digested remainder.

As used herein, the term “osmotic shock” refers to a change in osmotic pressure relative to the physiological osmotic pressure within the cell that causes damage to the cell. In some embodiments, the cells are damaged by osmotic shock by applying a medium or solution having osmolality properties falling outside a physiological range under conditions that induce osmotic shock to the cells. The medium or solution having osmolality properties falling outside a physiological range may be a hypotonic or hypoosmolar or hypoosmotic solution with lower osmolality than the physiological osmolality. The medium or solution having osmolality properties falling outside a physiological range may be a hypertonic or hyperosmolar or hyperosmotic solution with higher osmolality than the physiological osmolality. The medium or solution having osmolality properties falling outside a physiological range may be a solution of any kind. In some embodiments, the medium or solution having osmolality properties falling outside a physiological range may be water, ultrapure water, distilled water, 5% glucose solution, a high salt solution, and the like.

The osmotic shock may be induced by applying the medium or solution having osmolality properties falling outside a physiological range to the lumen in an amount which may lead the duodenum to distend, or by applying such medium or solution to the mucosal layer of a tissue. In some embodiments, the medium or solution having osmolality properties falling outside a physiological range may be in contact with the lumen or mucosal layer for about 0.5 minutes, 1 minute, about 2 minutes, about 5 minutes, about 10 minutes or about 15 minutes.

In some embodiments, the method of isolating one or more multipotent cells, or a population including such cells, expressing one or more desired biomarkers from a tissue having a mucosal layer and a submucosal layer comprises a further processing of the mixture into a cell suspension prior to the selection or isolation step. In some embodiments, the method of isolating one or more multipotent cells, or a population including such cells, expressing one or more desired biomarkers from a tissue having a mucosal layer and a submucosal layer comprises one or more wash steps using a physiologically acceptable medium.

The method of isolating one or more multipotent cells, or a population including such expressing one or more desired biomarkers from a tissue having a mucosal layer and a submucosal layer may be used to isolate multipotent stem cells from any suitable tissue having a mucosal layer and a submucosal layer. In some embodiments, the tissue is endodermal tissue. In some embodiments, the tissue is selected from the group comprising small intestine, large intestine, rectum. In some embodiments, the tissue is selected from the group comprising trachea, main bronchus, esophagus, stomach and duodenum.

In a particular aspect, this disclosure relates to a method of isolating one or more BGSCs or a population including BGSCs from a tissue, a portion of such tissue or a sample of same taken from a duodenum of a subject comprising:
removal of the intestinal mucus;
applying a medium or solution having osmolality properties falling outside a physiological range under conditions under conditions that induce osmotic shock to the cells of the mucosal layer;
removing or dissolving at least a portion of the mucosal layer or the cells thereof by mechanical, surgical and/or chemical methods, leaving and/or exposing a remainder which may include a submucosal layer;
applying to the mucosal layer and/or the remainder a medium or solution to substantially kill, inactivate or remove pathogens and/or pathogenic and/or beneficial microbes;
applying digestion or dissociation to the remainder, including the submucosal layer, to produce a digest, dissociated cellular material, or a cell suspension;
optionally culturing at least some of the digest, dissociated cellular material, or cells from the cell suspension; and
isolating those cells that express, or a population of cells in which at least some, or a substantial portion of, or a majority of the cells express, one or more of Tra-1-60, Tra-1-81, OCT4, SOX2, NANOG, EpCAM, SOX9, CK7, Lgr5, NIS, CD44, CK19; and/or those cells that express both SOX17 and PDX1.

As used herein, the term “remainder” refers to a tissue, a portion thereof, or sample of the same, with the mucosal layer partly or entirely disrupted and/or removed, so that the submucosal layer is exposed. The desired multipotent cells such as the BGSCs are contained within the remaining submucosal layer.

In some embodiments of the method of isolating one or more BGSCs, or a population including BGSCs, from a duodenum of a subject, a portion thereof or a sample of same, the removing step is carried out by chemical disruption, which comprises a use of an emulsifier and/or a detergent. In some embodiments, sanitization or sterilization is carried out with a sodium hypochlorite solution step (d). In some embodiments, digestion is carried out enzymatically. In some embodiments of the method of isolating one or more BGSCs or a population including BGSCs, the remainder after the removing or dissolving step comprises a submucosal layer and the digested remainder comprises tissue fragments. In some embodiments of the method of isolating one or more BGSCs or a population including BGSCs, the tissue or tissue portion or sample is minced before the digestion step (e). In some embodiments of the method of isolating one or more BGSCs or a population including BGSCs, the isolation step (f) is performed using culture selection with culture conditions comprising a serum-free medium, optionally, Kubota's Medium. In some embodiments of the method of isolating one or more BGSCs or a population including BGSCs, the isolation step (f) is performed using culture selection with culture conditions containing serum. In some embodiments of the method of isolating one or more BGSCs or a population including BGSCs, the digestion step (e) and/or the isolation step (f) is performed on low attachment plates. In some embodiments of the method of isolating one or more BGSCs, the isolated cells are cultured under suspension or 3D conditions that support or produce spheroids, one or more organoids, cell clusters, or cell aggregates.

The mechanical disruption/mucosectomy could be done by various possible procedures and tools which remove the mucosal layer. In some embodiments, the cell surface layer is peeled off. Many different methods of mechanical disruption of cell layers are known, such as using small beads to shear open the cell wall, using sonication to disrupt cell walls, using grinding by mortar and pestle, using blenders, using freezing and thawing cycles, using microwaves to disrupt the bonds within the cell walls and to denature the proteins, or using high pressure, and the like.

In some embodiments of the method of isolating one or more BGSCs or a population of cells including BGSCs, the removing or dissolution step is carried out by chemical disruption, which comprises use of an emulsifier selected from the group comprising Lecithins, Polyoxyethylene Sorbitan Monolaurate (Polysorbate 20), Polyoxyethylene Sorbitan Monooleate (Polysorbate 80), Polyoxyethylene Sorbitan Monopalmitate (Polysorbate 40), Polyoxyethylene Sorbitan Monostearate (Polysorbate 60), Polyoxyethylene Sorbitan Tristearate (Polysorbate 65), Ammonium Phosphatides, Sodium, Potassium and Calcium Salts of Fatty Acids, Magnesium Salts of Fatty Acids, Mono- and Diglycerides of Fatty Acids, Acetic Acid Esters of Mono- and Diglycerides of Fatty Acids, Lactic Acid Esters of Mono- and Diglycerides of Fatty Acids, Citric Acid Esters of Mono- and Diglycerides of Fatty Acids, Mono- and Diacetyl Tartaric Acid Esters of Mono- and Diglycerides of Fatty Acids, Mixed Acetic and Tartaric Acid Esters of Mono- and Diglycerides of Fatty Acids, Sucrose Esters of Fatty Acids, Sucroglycerides, Polyglycerol Esters of Fatty Acids, Polyglycerol Polyricinoleate, Propane-1,2-Diol Esters of Fatty Acids, Thermally Oxidised Soya Bean Oil Interacted with Mono- and Diglycerides of Fatty Acids, Sodium Stearoyl-2-Lactylate, Calcium Stearoyl-2-Lactylate, Sorbitan Monostearate, Sorbitan Tristearate, Sorbitan Monolaurate, Sorbitan Monooleate, and Sorbitan Monopalmitate.

In some embodiments of the method of isolating one or more BGSCs, the removing or dissolution step is carried out by chemical disruption, which comprises use of a detergent selected from a group comprising 1-Heptanesulfonic Acid; N-Laurylsarco sine, Lauryl Sulfate, 1-Octane Sulfonic Acid and Taurocholic Acid, Benzalkonium Chloride, Cetylpyridinium, Methylbenzethonium Chloride, Decamethonium Bromide, Alkyl Betaines, Alkyl Amidoalkyl Betaines, N-Dodecyl-N,N-Dimethyl-3-Ammonio-1-Propanesulfonate, Phosphatidylcholine, N-Decyl A-D-Glucopyranoside, N-Decyl A-D-Maltopyranoside, N-Dodecyl B-D-Maltoside, N-Octyl B-D-Glucopyranoside, N-Tetradecyl B-D-Maltoside, Tritons (Triton X-100), Nonidet-P-40, Poloxamer 188, Sodium Lauryl Sulfate, Sodium Deoxycholate, and Sodium Dodecyl Sulfate.

In one aspect, the present disclosure relates to a method of isolating one or more BGSCs or a cell population that includes BGSCs from a tissue sample taken from a duodenum of a subject comprising:
contacting the mucosal layer with a solution having osmolality properties falling outside a physiological range under conditions under conditions to induce osmotic shock to the cells of the mucosal layer
removing or dissolving at least a portion of the mucosal layer or the cells thereof by mechanical, surgical and/or chemical methods, leaving and/or exposing a remainder, which may include a submucosal layer;
contacting the remainder with a medium or solution to substantially kill, inactivate, or remove pathogens and/or pathogenic and/or beneficial microbes;
digesting or dissociating the remainder to provide a cell suspension;
optionally culturing at least some of the cells from the digest or dissociated cellular or tissue material; and
isolating one or more multipotent cells which express, or a population of cells in which at least some, or a substantial portion of, or a majority of the cells express, one or more desired biomarkers.

In some embodiments, mucus is removed from the tissue or tissue portion or sample prior to contacting the mucosal layer with a solution having osmolality properties falling outside a physiological range under conditions to induce osmotic shock to the cells of the mucosal layer.

In another aspect, this disclosure relates to a method of isolating BGSCs from a duodenum, a portion thereof, or a sample taken from same comprising:
digesting or dissociating a duodenum, a portion thereof, or a sample taken from same to provide a digest or dissociated cellular material;
obtaining from the digested or dissociated cellular material: (i) those cells that express, or a population of cells in which at least some, a substantial portion, or a majority of the cells expresses, one or more of Tra-1-60, Tra-1-81, OCT4, SOX2, NANOG, EpCAM, SOX9, CK7, Lgr5, NIS, CD44 and CK19; and/or (ii) those cells that express, or a population of cells in which at least some, a substantial portion, or a majority of the cells express, both SOX17 and PDX1.

In some embodiments of the method of isolating BGSCs or a population of cells including BGSCs, the duodenum, a portion thereof, a sample taken from same, the remainder and/or the digest or dissociated cellular or tissue material, or combinations thereof, are contacted with a disinfectant or sanitizing medium, solution or agent(s).

As used herein, the term “disinfectant” is contemplated to include all mediums, solutions or agents that destroy pathogens and/or pathogenic and/-or beneficial microbes such as bacteria, viruses and fungi, and also include mediums, solutions or agents that will kill bacterial or fungal spores. In some embodiments, the disinfectant is a hypochlorite solution. In some embodiments, the disinfectant is a solution with from about 0.01% to about 0.1% sodium hypochlorite, or from about 0.1% to about 0.2% sodium hypochlorite. In some embodiments, the disinfectant is a 0.01% sodium hypochlorite solution, 0.02% sodium hypochlorite solution, 0.05% sodium hypochlorite solution, 0.1% sodium hypochlorite solution, 0.15% sodium hypochlorite solution, or 0.2% sodium hypochlorite solution. In a preferable embodiment, the disinfectant is a 0.05% sodium hypochlorite solution.

Alternatively, in some embodiments, the disinfectant may be a medium, solution or agent selected from the group comprising alcohol, sodium hydroxide, aldehydes, oxidixing agents, peroxy and peroxo acids, phenolics, quarternary ammonium compounds, inorganic compounds (such as chlorine, iodine, acids and bases, metals), or terpenes, etc. As used herein, disinfectants also include antibiotics such as penicillins, polymyxins, rifamycins, lipiarmycins, quinolones, or sulfonamides, etc.

It is a discovery of the present disclosure that contacting the duodenum, a portion thereof, or a sample taken from same with a disinfectant or sanitizing medium, solution or agent results in the obtained BGSCs of population of cells including BGSCs is substantially free of pathogens and/or free of pathogenic and/or beneficial microbes. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, in some embodiments, the term “substantially free of pathogens and/or free of pathogenic and/or beneficial microbes” may refer to a situation in which the presence of pathogens and/or pathogenic and/or beneficial microbes is at a level that may be acceptable for the desired use or may not prevent the desired use of the BGSCs or population of cells including BGSCs, or at a level that undetectable in the sample of interest as determined by commonly known methods. Such methods include for example standard sterility tests for gram+, gram−, aerobic and anaerobic bacteria, mycoplasm and endotoxin tests. The same applies to the term “substantially free of pathogens and/or free of pathogenic and/or beneficial microbes” in connection with cells or populations of cells other than BGSCs obtained from a tissue, or tissue portion or sample, according to the methods of this disclosure.

Accordingly, compositions of this disclosure may comprise BGSCs or a cell population including BGSCs which are substantially free of pathogens and/or free of pathogenic and/or beneficial microbes, expressing one or more of the markers Tra-1-60, Tra-1-81, OCT4, SOX2, NANOG, EpCAM, SOX9, CK7, Lgr5, NIS, CD44, CK19 and both SOX17 and PDX1.

Compositions of this disclosure may also comprise isolated BGSCs or other multipotent cells, or a cell population including such cells, in combination with a physiologically acceptable medium. As used herein, the term “physiologically acceptable medium” refers to any medium that conventional pharmaceutical practices use for formulating pharmaceutical compositions for administration to a subject such as a human patient. The physiologically acceptable medium may comprise physiological saline or an isotonic solution containing glucose and other supplements such as carbohydrates such as glucose, mannose, sucrose or dextrans, mannitol; proteins; polypeptides or amino acids such as glycine; antioxidants; vitamins, chelating agents such as EDTA or glutathione; adjuvants (e.g., aluminum hydroxide); and preservatives. Compositions of the present invention may be formulated for injection of the BGSCs or cell population including BGSCs into the circulation of a subject or directly into a target organ or tissue.

Accordingly, this disclosure relates to a composition of BGSCs or a population including BGSCs substantially free of pathogens and/or free of pathogenic and/or beneficial microbes, expressing one or more of Tra-1-60, Tra-1-81, OCT4, SOX2, NANOG, EpCAM, SOX9, CK7, Lgr5, NIS, CD44, CK19 and both SOX17 and PDX1, and a physiologically acceptable medium.

Methods of Using the Isolated Brunner's Gland Stem/Progenitor Cell (BGSC)

The BGSCs and/or population of cells including BGSCs disclosed herein is contemplated for use in medical treatment.

For example, this disclosure relates to a method of treating a subject diagnosed with a disease or condition involving or affecting liver, pancreas, stomach, duodenum, small intestine, large intestine, rectum and/or other endodermal tissue, comprising administering to a subject in need thereof an effective amount of a population of BGSCs.

In some embodiments, this disclosure relates to a method of treating a subject diagnosed with a disease or condition involving or affecting liver, pancreas, stomach, duodenum, small intestine, large intestine, rectum and/or other endodermal tissue comprising the administration of an effective amount of a population of cells including at least some, or a substantial portion of, or a majority of BGSCs expressing one or more of the markers selected from the group consisting of Tra-1-60, Tra-1-81, OCT4, SOX2, NANOG, EpCAM, SOX9 and CK7, Lgr5, NIS, CD44, CK19 and both SOX17 and PDX1.

In another aspect, this disclosure relates to a method of autologous cell or gene therapy comprising the administration of an effective number of BGSCs, or population of cells including at least some, or a substantial portion of, or a majority of BGSCs, which express one or more of the markers selected from the group consisting of Tra-1-60, Tra-1-81, OCT4, SOX2, NANOG, EpCAM, SOX9 and CK7, Lgr5, NIS, CD44, CK19 and both SOX17 and PDX1, and which are further characterized as capable of proliferation, with limited or minimal differentiation, under culture conditions that support self-renewal. In some embodiments of the method of autologous cell or gene therapy, the cells are genetically engineered or modified cells.

In another aspect, this disclosure relates to a method of allogeneic cell or gene therapy comprising the administration of an effective number of BGSCs or a population of cells including at least some, or a substantial portion of, or a majority of BGSCs which express one or more of the markers selected from the group consisting of Tra-1-60, Tra-1-81, OCT4, SOX2, NANOG, EpCAM, SOX9 and CK7, Lgr5, NIS, CD44, CK19 and both SOX17 and PDX1, and which are further characterized as capable of proliferation, with limited or minimal differentiation, under culture conditions that support self-renewal. In some embodiments of the method of allogeneic cell or gene therapy, the cells are genetically engineered or modified cells.

In another aspect, this disclosure relates to a use of BGSCs which express one or more of the markers selected from the group consisting of Tra-1-60, Tra-1-81, OCT4, SOX2, NANOG, EpCAM, SOX9 and CK7, Lgr5, NIS, CD44, CK19 and both SOX17 and PDX1 and which are further characterized as capable of proliferation, with limited or minimal differentiation, under culture conditions that support self-renewal for treatment of a disease or condition involving or affecting liver, pancreas, stomach, duodenum, small intestine, large intestine, rectum and/or other endodermal tissue and/or use for autologous or allogeneic cell or gene therapy, optionally with cells which are genetically engineered or modified.

In another aspect, this disclosure relates to a use of population of cells including at least some, or a substantial portion of, or a majority of BGSCs, expressing one or more of the markers selected from the group consisting of Tra-1-60, Tra-1-81, OCT4, SOX2, NANOG, EpCAM, SOX9 and CK7, Lgr5, NIS, CD44, CK19 and both SOX17 and PDX1 for treatment of a disease or condition involving or affecting liver, pancreas, stomach, duodenum, small intestine, large intestine, rectum and/or other endodermal tissue and/or use for autologous or allogeneic cell or gene therapy, optionally with cells which are genetically engineered or modified.

As used herein, “treating” or “treatment” of a disease in a subject refers to (1) preventing the symptoms or disease from occurring in a subject that does not yet display symptoms of the disease or displays limited symptoms; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation, amelioration or cessation of one or more symptoms, diminishment of extent of a condition (including a disease), stabilized (i.e., not worsening) state of a condition (including disease), delay or slowing of condition (including disease) progression, amelioration or palliation of the condition (including disease) states and remission (whether partial or total), whether detectable or undetectable.

As used herein the term “amount effective” or “effective amount” refers to an amount that is sufficient to treat the disease or condition being addressed. An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions for any particular patient may depend upon a variety of factors including the activity of the specific agent employed, the age, body weight, general health, co-mobidities, sex, and diet of the patient, the time of administration, the rate of metabolism and/or excretion, the composition combination, severity of the particular disease or condition (e.g. liver disease) being treated and form of administration.

### Modes of Carrying Out the Disclosure

This disclosure herein demonstrates that: i) a human and/or animal duodenum contains cells, including within Brunner's Glands, which are referred to herein as Brunner's Gland stem/progenitor cells (BGSCs), with the phenotypic traits of endodermal stem/progenitor cells positive for pluripotency or multipotency markers and other biomarkers of stem cells or “stemness”; ii) these cells have a distinct phenotype from that of intestinal stem cells within mucosal crypts, including, but not limited to, Tra-1-60, Tral-81, OCT4 and CK7 expression; iii) these cells are also have distinct phenotype from that of hepatic stem cells (which express SOX17 but not PDX1) and from that of pancreatic stem cells (which express PDX1 but not SOX17), as BGSCs may express both SOX17 and PDX1; iv) BGSCs may be isolated by chemical, mechanical and/or surgical procedures or methods which destroy the mucosal epithelial cells (villi and crypts) at least in part, but maintain the submucosal layer at least in part; v) BGSCs can be selected in vitro, where they display self-renewal properties, capability to form and grow as sphereoids, organoids, cell aggregates or cell clusters, and exhibit multipotency; vi) in vivo, BGSCs are able to engraft into tissues and differentiate into lineages associated with such tissues, for example engrafting into the liver of SCID mice and differentiating towards mature hepatocytes.

Brunner's glands are unique mucinous glands located within the submucosal layer in the duodenum. These glands have not been found in the stomach, nor in the other portions of the small intestine (i.e. jejunum and ileum) nor in the large intestine. Their primary known functions reside in the production of mucus which protects the duodenal mucosa from the acidity of materials coming from the stomach. The number of Brunner's glands progressively decreases from pyloric orifice towards duodeno-jejunal flexure, almost disappearing in the inferior and ascending duodenal portions. In the duodenal wall, Brunner's glands are separated from intestinal crypts (or glands) by muscolaris mucosae, but they are in direct anatomical continuity with them. Intestinal crypts contain a specific population of stem cells implicated in the continuous renewal of intestinal epithelium, following a crypt-to-villus axis.

The data provided herein establishes that, beside mucinous cells, Brunner's glands harbor a population of cells expressing a specific constellation of stem cell markers, such as SOX9, Lgr5, EpCAM, CD44, and/or both SOX17 and PDX1. These cells are small, with scant cytoplasm and with a high nucleus-to-cytoplasm ratio, and their phenotype is compatible with the profile of the ventral endoderm in embryos. Moreover, a restricted sub-population of these cells (nearly 5%) express markers of pluripotency such as Oct4A, SOX2, Tra-1-60, and Tra-1-81. Furthermore, BGSCs showed the expression of the proliferating marker PCNA thus indicating their replicative activity, probably implicated in the renewal of mucin-producing cells. Interestingly, the expression of stem cell markers was precisely distributed: pluripotency makers were expressed in cells located in deeper acini, while Lgr5 and PCNA were in cells near the muscolaris mucosae and in continuity with intestinal crypts. This distribution suggests the presence of two different but overlapping BGSC populations: one population has a primitive phenotype, is quiescent and located deeply within the submucosa; the other shows transit-amplifying features, crosses the muscolaris mucosae, and is spatially associated with intestinal crypts. However, both the “quiescent” and the transit-amplifying BGSC populations showed a phenotype (Lgr5^{+/-}/CK7⁺/CK19⁺/Tra-1-60⁺) that clearly distinguished them from cells of the intestinal crypts (Lgr5⁺/CK7⁺/CK19⁺/Tra-1-60-).

Aspects that are disclosed herein relate to an approach that has been developed to isolate BGSCs, or a population with at least some, or a substantial portion of, or majority of BGSCs, from human duodenum, including: chemical, mechanical or surgical disruption of the mucosal layer to at least partly eliminate the surface epithelium, leaving a remainder which may expose the submucosal layer; digestion or dissociation of the submucosal layer; isolation of cells, or a population of cells that includes cells, which express the markers described in this disclosure; This method my include culture selection conditions such as cultures of spheroids, organoids, cell aggregates or cell clusters maintained in serum-free Kubota's Medium. Once isolated, the cells in vitro matched the phenotype described in the organ (e.g., Lgr5^{+/-}/CK7⁺/CK19⁺/Tra-1-60⁺/pluripotency genes⁺), confirming the depletion of intestinal stem cells from the cell preparation. In vitro, BGSCs were able to grow as spheroids, organoids, cell aggregates or cell clusters and maintain their undifferentiated phenotype with null expression of mature cell markers and no evidence of mucin production. Remarkably, BGSCs were able rapidly to mature towards various fates, including at least towards hepatocytic, cholangiocytic and endocrine pancreatic lineages, when transferred into specific differentiation conditions.

Interestingly, a previous report indicated that human gastric epithelial and duodenal cells did not show stem cell behavior and multipotency (see table) without reprogramming. The results disclosed herein demonstrate, to the contrary, that stem/progenitor cells (BGSCs) are found within the human and animal duodenum, including the Brunner's glands. Given their position within the organ, these BGSCs or a cell population including these BGSCs are readily isolatable using the methods described in this disclosure, and do not need reprogramming but intrinsically show endodermal stem/progenitor features, character and capacities, and have multipotent properties.

In this study, the capability of differentiation towards mature endodermal fates has been tested. For example, BGSCs have been injected into murine livers via a vascular route.

In the field of liver diseases, orthotopic liver transplantation currently represents the only curative treatment for acute liver failure and end-stage chronic liver disease. Since liver transplantation is limited by a severe shortage of organ donors, cell therapy strategies could represent a feasible alternative option to support liver functions while waiting for organ allocation. However, regenerative medicine approach for liver diseases requires the identification of sustainable and readily available cell sources.

This disclosure provides a novel stem cell niche with multipotent capability and the procedures to isolate the applicable cells from postnatal duodenum. Human BGSCs represent a potential readily available source obtainable from human donors. The cells do not require genetic reprogramming or major manipulation and should be more easily usable (and potentially a safer approach) in clinical programs compared to reprogrammed cells. Moreover, they have the unique potential as a cellular source that can be retrieved using endoscopy and then used for autologous or allogeneic cell and gene therapies.

### Abbreviations

AFP, α-fetoprotein; ALB, albumin; BTSCs, biliary tree stem cells; CD, common determinant; CD44, hyaluronan receptors; CD133, prominin; CFTR, cystic fibrosis transmembrane conductance regulator; cGMP, current good manufacturing practices; CK, cytokeratin protein; CXCR4, CXC-chemokine receptor 4 (also called Fusin or CD184; also called platelet factor 4; E).4 PI, 6-diamidino-2-phenylindole; DPBS, Dulbecco's Phosphate Buffered Saline; EGF, epidermal growth factor; EpCAM, epithelial cell adhesion molecule; FBS, fetal bovine serum (or FCS, fetal calf serum) FGF, fibroblast growth factor (FGF 10 is one of the many forms of FGF); HBs, hepatoblasts; HDM, hormonally defined medium; HDM-C, an HDM designed to lineage restrict cells to cholangiocytes; HDM-H, an HDM designed to lineage restrict cells to hepatocytes; HDM-P, an HDM designed to lineage restrict cells to a pancreatic fate; HGF, hepatocyte growth factor; HpSCs, hepatic stem cells; IF, immunofluorescence; IHC, immunohistochemistry; KM, Kubota's Medium, a serum-free medium designed for endodermal stem cells; KPT, cytokeratin gene; Lgr5, Leucine-rich repeat-containing G-protein coupled receptor 5 that binds to R-spondin; MKM, modified Kubota's Medium consisting of Kubota's Medium supplemented with calcium, copper, and bFGF; NANOG, a transcription factor critically involved with self-renewal; NCAM, neural cell adhesion molecule; NIS, sodium/iodide symporter; OCT4, (octamer-binding transcription factor 4) also known as POU5F1 (POU domain, class 5, transcription factor 1), a gene expressed by stem cells; PBS, phosphate buffered saline; PDX1, pancreatic and duodenal homeobox 1, a transcription factor critical for pancreatic development; PBGs, peribiliary glands, stem cell niches for biliary tree stem cells; RMPI, Roswell Memorial Park Institute the acronym is used for various basal media established by investigators at the institute; RT-PCR, Reverse-transcription polymerase chain reaction; SALL4, Sal-like protein 4 found to be important for self-replication of stem cells; SOX, Sry-related HMG box; SOX2, a transcription factor that is essential for maintaining self-renewal, or pluripotency in embryonic and determined stem cells. SOX9, transcription factor associated with endodermal tissues (liver, gut, biliary tree and pancreas); SOX17, a transcription factor essential for differentiation of liver; VEGF, vascular endothelial cell growth factor.

Materials and Methods

Human Tissue Sourcing

Human duodena were obtained from organ donors from the “Paride Stefanini” Department of General Surgery and Organ Transplantation, Sapienza University of Rome, Rome, Italy. Informed consent to use tissues for research purposes was obtained from our transplant program. Protocols received Institutional Review Board approval, and processing was compliant with current Good Manufacturing Practice (cGMP). The research protocol was reviewed and approved by the Ethic Committee of Umberto I Policlinico of Rome.

Media and Solutions

All media were sterile-filtered (0.22-μm filter) and kept in the dark at 4° C. before use. RPMI-1640, the basal medium for all the cell cultures, and fetal bovine serum (FBS) were obtained from GIBCO/Invitrogen (Carlsbad, Calif.). All reagents were obtained from Sigma (St. Louis, Mo.) unless otherwise specified. Growth factors, except those noted, were purchased from R&D Systems (Minneapolis, Minn.).

Kubota's Medium (KM) consists of any basal medium (here being RPMI 1640) with no copper, low calcium (0.3 mM), 10⁻⁹ M Selenium, 0.1% bovine serum albumin (BSA), 4.5 mM Nicotinamide, 0.1 nM Zinc Sulfate heptahydrate, 10⁻⁸ M hydrocortisone (or dexamethasone), 5 μg/ml transferrin/Fe, 5 μg/ml insulin, 10 μg/ml high density lipoprotein, and a mixture of free fatty acids that are added bound to purified human serum albumin. The detailed protocol of its preparation was first reported by Kubota and Reid as a defined medium for hepatoblasts². Kubota's Medium has since been shown effective for murine, rodent, and human hepatic stem cells, biliary tree stem cells, hepatoblasts, gall bladder-derived stem cells and pancreatic progenitors³⁻⁹.

For differentiation studies, serum-free KM was supplemented with calcium (final concentration: 0.6 mM), copper (10⁻¹² M) and 20 ng/ml bFGF and referred to as modified Kubota's Medium (MKM). MKM was used as a base and with specific supplements to prepare different hormonally defined media (HDM) used to induce selective differentiation of BG cells towards hepatic (HDM-H) versus pancreatic islet (HDM-P) fates:
HDM-H for hepatic differentiation: was prepared supplementing MKM with 7 μg/L glucagon, 2 g/L galactose, 1 nM triiodothyroxine 3 (T3), 10 ng/ml Oncostatin M (OSM); 10 ng/ml epidermal growth factor (EGF), 20 ng/ml hepatocyte growth factor (HGF), and 1 μm dexamethasone.
HDM-P for Pancreatic islet cell differentiation: MKM without hydrocortisone, supplemented with 2% B27, 0.1 mM ascorbic acid, 0.25 μM cyclopamine, 1 μM retinoic acid; bFGF was added for the first 4 days and then replaced with 50 ng/ml exendin-4 and 20 ng/ml of HGF.

Magnetic Sorting Procedures

Cells were sorted for EpCAM or TRA-1-60 by using magnetic bead immunoselection by a protocol specified by the manufacturer (Miltenyi Biotec Inc., Germany). Briefly, the positive cells were magnetically labeled with EpCAM MicroBeads (Miltenyi Biotec Inc., catalog #130-061-101) or with TRA-1-60 MicroBeads (Miltenyi Biotec Inc., catalog #130-100-832). Then, the cell suspension was loaded onto a MACS LS Column (Miltenyi Biotec Inc., catalog #130-042-401) that was placed in the magnetic field of a MACS Separator. The magnetically labeled cells were retained within the column while the unlabeled cells ran through. After removing the column from the magnetic field, the magnetically retained cells were eluted as a positively selected cell fraction. The positive cells were evaluated by cell count and cell viability as previous described. Positive cells were suspended in basal medium at a concentration of 300,000 cells per ml, and used as the final cell suspension. N. 4 aliquots, containing approximately 200,000 cells, were collected for flow cytometry.

Cell Isolation Under GMP Conditions and Sterility Testing

To produce BG stem/progenitor cells in cGMP conditions for future clinical application, duodena were processed following “The rules governing medicinal products in the European Union” and the European guidelines of good manufacturing practices for medicinal products for human use (EudraLex—Volume 4 Good manufacturing practice Guidelines). Sterility testing was performed under cGMP conditions by a “direct inoculation method” and in accordance with guidelines of good manufacturing practices for medicinal products for human and veterinary use.

Cell Cultures and Clonal Expansion

Unsorted and sorted cells (approximately 3×10⁵), obtained from duodenal specimens, were seeded onto 3 cm diameter plastic culture dishes and kept overnight (12 hours) in KM with 10% FBS. Thereafter cell cultures were maintained in serum-free KM and observed for at least 2 months. For testing clonal expansion, a single cell suspension was obtained and cells were plated at a clonal seeding density of 500 cells/cm² in serum-free KM, a self-replication medium.

Organoid Preparation and Culture

After centrifugation, the cell pellets were suspended in KM and 3×10⁵ cells were placed onto 12-wells 2.2 cm diameter plastic culture dishes and kept overnight (˜12 hours) in KM with 10% FBS; thereafter, the cultures were provided with serum-free KM. The cells were cultivated in KM in an incubator at 37° C., with atmospheric oxygen and 5% CO₂ for 1 week allowing one to obtain more cell population. After 7 days the cells were removed from 12-well plates, and the pellet of cells was embedded in 400 μl of cold Matrigel (Corning Matrigel Basement Membrane Matrix Growth Factor Reduced, phenol red-free). Applicants seeded a volume of 400 μl of gel, containing 2×10⁵ cells per 12-well plate. Following polymerization (15 min, 37° C.), the gels were overlaid with 500 μl of organoid culture medium. Organoid culture medium was based on Ad-DMEM/F12 (Life Technologies) supplemented with B27, N2 (Life Technologies), and 1.25 mM N-acetylcysteine (Sigma-Aldrich), 10 nM gastrin (Sigma-Aldrich), and the growth factors: 50 ng/ml EGF (Peprotech), 1 μg/ml Recombinant Human R-Spondin-1 (Perotech), 100 ng/ml FGF10 (Peprotech), 25 ng/ml HGF (Peprotech), 10 mM Nicotinamide (Sigma-Aldrich), 5 μM A83-01 (Tocris), and 10μM Forskolin (FSK). Applicants changed the medium every 2-3 days, controlling the size and number of organoids microscopically.

After 10-14 days, organoids were removed from the Matrigel, using Cell recovery solution (Corning) and ice-cold PBS. The organoids in culture gels were gently disrupted with Cell recovery solution (Corning) to break the Matrigel into small fragments, while preserving organoids as whole spheres. The organoids were then gently centrifuged to obtain intact organoids collected at the bottom of the tube. Most of the supernatant was removed with a pipette, and the organoid pellets were fixed with 4% formalin for further analysis.

Positive Controls

NTERA-2 clone D1 pluripotent human embryonic cell line (Sigma Aldrich, St. Louis, Mo., USA; code: 01071221) was used as positive controls for pluripotency markers (SOX2, OCT4A and NANOG), for flow cytometry, cell culture and RT-PCR experiments¹⁰. Moreover, fragments of human seminoma testis have been used as positive controls for immunohistochemistry experiments on pluripotency markers.

HT-29, a human colon adenocarcinoma cell line (LGC Standards S.r.L, Milan, Italy; code: ATCC-HTB-38) was used as positive control for Lgr5 antibody for flow cytometry and RT-PCR experiments. Normal pancreatic islet cells have been used as a control for experiments on pancreatic islet differentiation and were purchased from ProdoLab, Irvine Calif. US (HIR-001)

Primary human hepatocytes (Clonetics™ Human Hepatocyte Cell Systems NHEPS™ Cells, code: CC-2591S) have been purchased commercially from Lonza (Basel, Switzerland) and used as a positive control for experiments on hepatocyte differentiation.

Normal pancreatic islet cells have been used as a control for experiments on pancreatic islet differentiation and were purchased from ProdoLab, Irvine Calif. US (HIR-001).

Light Microscopy (LM), Immunohistochemistry (IHC) and Immunofluorescence (IF)

Specimens were fixed in 10% buffered formalin for 2-4 hours, embedded in low-temperature-fusion paraffin (55-57° C.), and 3-4 μm sections were stained with hematoxylin-eosin and Sirius red/Fast green, according to standard protocols. For IHC, endogenous peroxidase activity was blocked by a 30 min incubation in methanolic hydrogen peroxide (2.5%). Antigens were retrieved, as indicated by the vendor, by applying Proteinase K (Dako, code S3020) for 10 min at room temperature. Sections were then incubated overnight at 4° C. with primary antibodies (Supplementary Table 1). Samples were rinsed twice with PBS for 5 min, incubated for 20 min at room temperature with secondary biotinylated antibody (LSAB+ System-HRP, Dako, code K0690; Glostrup, Denmark) and then with Streptavidin-HRP (LSAB+ System-HRP, Dako, code K0690). Diaminobenzidine (Dako) was used as a substrate, and sections were counterstained with hematoxylin. For immunofluorescence on cell culture, slides chambers were fixed in acetone for 10 min at room temperature and then rinsed with PBS-Tween 20. Non-specific protein binding was blocked by 5% normal goat serum. Fixed cells were incubated with primary antibodies. Then, cells were washed and incubated for 1 h with labeled isotype-specific secondary antibodies (anti-mouse AlexaFluor-546, anti-mouse Alexafluor-488, anti-rabbit Alexafluor-488, anti-goat AlexaFluor-546, Invitrogen, Life Technologies Ltd, Paisley, UK) and counterstained with 4,6-diamidino-2-phenylindole (DAPI) for visualization of cell nuclei. For all immunoreactions, negative controls were also included and consisted of replacing the primary antibody with pre-immune serum. Sections/Cultures were examined in a coded fashion by Leica Microsystems DM 4500 B Light and Fluorescence Microscopy (Weltzlar, Germany) equipped with a Jenoptik Prog Res C10 Plus Videocam (Jena, Germany). IF staining were also analyzed by Confocal Microscopy (Leica TCS-SP2). LM, IHC and IF observations were processed with an Image Analysis System (IAS—Delta Sistemi, Roma-Italy) and were independently performed by two researchers in a blind fashion.

The area occupied by BGs was evaluated by an Image Analysis System (IAS—Delta Sistemi, Rome—Italy). Using it, Applicants determined that the volume occupied by BGs has been calculated as the total area occupied by the glandular acini and expressed as the percentage with respect to the total duodenal submucosa. All counts have been performed in six non-overlapping fields (magnification ×20) for each slide; at least 3 different slides have been taken from each specimen.

For IHC/IF staining, the number of positive cells was counted in a random, blinded fashion in six non-overlapping fields (magnification ×20) for each slide/culture, and the data are expressed as % positive cells. IF stainings were also scanned by a digital scanner (AperioScanscope FL System, Aperio Technologies, Inc, Oxford, UK) and processed by ImageScope. An image analysis algorithm has been used to quantify the proportion of positive pixel area for single fluorophore or the area with co-localization of two fluorophores. To test the glycogen-storage capability, a Periodic Acid-Schiff (PAS) staining system (Sigma Aldrich, INC, Catalog No. 395) and α-amylase (Sigma Aldrich, INC, Catalog No. A 3176) digestion procedure (followed by the PAS stain) has been used according to the manufacturer's procedure.

Flow Cytometry (FC) Analysis

Cells in culture were trypsinized, dissociated by gentle pipetting and suspended at approximately 2×10⁵ cells/ml in PBS. Isolated cells were labeled with fluorescent primary antibodies or isotype controls. For intracellular antigens, cells were fixed in 4% paraformaldehyde and permeabilized with PBS-Saponin 0.5%—FCS 10%, prior to incubation with the primary antibody. Primary antibodies included EpCAM (EpCAM-FITC, MiltenyiBiotec Inc., catalog #130-080-301), Lgr5 (Lgr5-PE, Origene Technologies Inc., Rockville, Md., USA catalog #TA400001), TRA-1-60 (TRA-1-60-PE, MiltenyiBiotec Inc., catalog #130-100-347). Cells were analyzed by a BD FACScanto™ Flow Cytometer (Becton, Dickinson and Company, NJ, USA). Ten thousand events were acquired and analyzed by BD FACSDiva™ software (Becton, Dickinson and Company, NJ, USA).

Reverse-Transcription Polymerase Chain Reaction (RT-PCR) Analysis

The RNA extractions were performed on tissues or cultures maintained for 6 days in serum-free KM and then with an additional seven days incubation (13-days total) in either the KM or one of the HDMs. Total RNA was extracted by the procedures of Chomczynski and Sacchi¹¹. RNA quality and quantity were evaluated with the Experion Automated Electrophoresis System RNA equipped with the RNA StSens Analysis Chip (Bio-Rad Laboratories, Hercules, Calif., USA) as previously described. The RNA extractions were performed on cultures maintained for 6 days in serum-free KM and that underwent an additional seven days' incubation (13-days total) in either the KM or one of the HDMs. The expression of albumin (ALB), cytochrome P450 (CYP3A4), insulin (INS), Glucagon (GLUC), PDX-1, SOX17, OCT4A, SOX2, and NANOG genes was conducted by reverse-transcription and PCR amplification performed in a closed tube (OneStep RT-PCR by Qiagen, Hamburg, Germany) on total RNA samples extracted from cells and tissues. These genes were co-amplified with the GAPDH housekeeping gene used as a reference. The gene expression was measured by the quantification of amplicons with on-chip capillary micro-electrophoresis performed with the Experion System (Bio-Rad, UK). The expression of the gene of interest was calculated by the ratio of the concentrations of the gene of interest and the reference gene GAPDH (reported by instrument in nmol/L) (Supplementary Table 2).

In Vivo Transplantation of BGSCs into Livers of Normal Mice

Five SCID (severe combined immunodeficiency) male mice were housed in a room at a mean constant temperature of 22° C. with a 12-h light-dark cycle, and free access to standard pellet chow and water. Study protocols were performed in compliance with our institutional guidelines. Experimental procedures were approved by the Ethical Committee on Animal Experiments of the EU Directive 2010/63/EU of Sapienza University of Rome and Umberto I University Hospital of Rome (Prot.#: 541). Suspensions of 2×10⁶ of human BGSCs in 100 μl saline were injected into the liver via the splenic artery. Sham controls mice were infused only with 100 μl saline. All the animals were closely monitored until recovery, and were allowed free access to food and water. No mortality was observed.

One month after transplantation, animals were sacrificed, and their livers were harvested. Liver fragments were placed in 10% buffered formalin for histology and immunohistochemistry and in Trizol reagent for gene expression analysis. Necrosis and fibrosis were evaluated respectively in Hematoxylin and Eosin (H&E) and Sirius Red stains. The human BGSC engraftment in murine livers, and their differentiation was assessed by immunohistochemistry for anti-human antibodies (anti-human mitochondria, anti-human HepPar-1, anti-human albumin) which do not react with mouse antigens as described elsewhere.

Immunohistochemistry stained (anti-human mitochondria) slides were scanned by a digital scanner (Aperio Scanscope CS System, Aperio Technologies, Inc, Oxford, UK) processed by ImageScope. An image analysis algorithm was to quantify the proportion of the area occupied by anti-human mitochondria-positive cells.

The RT-PCR for human albumin in mice was performed as previously described. Briefly, specific primers for human albumin (Supplementary Table 2) were designed as programmable specific sequence to discriminate specifically the human albumin gene from the murine gene, by using the Universal Probe Library Assay Design Center (Roche).

Statistical Analysis

Data are expressed as mean±standard deviation (SD). Statistical analyses were performed by SPSS statistical software (SPSS Inc. Chicago Ill., USA). Differences between groups for not normally distributed parameters were tested by Mann-Whitney U tests. Statistical significance was set to a p-value <0.05.

### Example 1—Isolation of Cells from Mucosa

Human duodena comprising hepato-pancreatic ampulla and pancreas were obtained from organ donors from the “Paride Stefanini” Department of General Surgery and Organ Transplantation, Sapienza University of Rome, Rome, Italy. Informed consent to use tissues for research purposes was obtained through our transplant program. All samples derived from adults between the ages of 19 and 73 years. Protocols received the approval of our Institutional Review Board, and processing was compliant with current Good Manufacturing Practice (cGMP). The research protocol was reviewed and approved by the Ethic Committee of Umberto I University Hospital, Rome. A human duodenum was carefully separated from the pancreas, and the intestine containing the hepato-pancreatic ampulla was removed surgically. The duodenum was cut into slices with a scalpel. Thereafter tissue specimens were processed as previously described. In brief, tissues were digested in RPMI 1640 supplemented with 0.1% bovine serum albumin, 1 nM selenium, antibiotics, type I collagenase (300 collagen digestion unit/nil) (Sigma-Aldrich Italy), 0.3 mg/ml deoxy-ribonuclease (Sigma-Aldrich, Italy), at 37° C. with frequent agitation for 30-45 min. Suspensions were filtered through an 800 micron metallic mesh filter (IDEALE ACLRI9 inox stainless steel) and spun at 270 g for 10 min before resuspension. Thereafter, cell suspensions were passed consecutively through 100 and 30 micron mesh filters; then, cell counting was carried-out by Fast-Read 102 (Biosigma Srl, Venice, Italy) and cell viability by the Trypan Blue assay (expressed as % of viable cells over total cells). The same method previously developed and utilized successfully for the liver and the biliary tree, when employed in the human duodenum, instead resulted in the creation of macro-aggregates. These macro-aggregates contained coated and crushed cells, which constantly (N=10) led to non-viable isolated cells and made it impossible to obtain cell cultures. It was speculated that this is due to the physical and chemical properties of the digested tissues which are highly saturated with mucus and degradation products released by mucosal epithelial cells, and that this leads to a sort of molecular web incorporating cells which are crushed during the procedure. Indeed, methods established in animals or humans to isolate cells from intestine avoid the destruction of the mucosal epithelia.

Another feature of cultures obtained by this method was frequent contamination (6/10). Because of these results, a strategy adopted was to separate the mucosal epithelia from the sub mucosa in order to keep the Brunner glands and avoid the problems of microbial contamination described above. Four different strategies were tried: 1) surgical dissection (N=3), 2) mucosectomy by previous injection of normal saline under the mucosa (N=3), 3) scraping the mucosa (N=3), 4) selective solubilization of the mucosa (N=10). All the methods proved illogical and yielded identical results to the initial method with the exception of the selective solubilization of the duodenum mucosa by a specific detergent solution injected into the intestinal lumen.

Unsuccessful and Suboptimal Isolation Procedures

A human duodenum was carefully separated from the pancreas, and the entire part of the intestine containing the hepato-pancreatic ampulla was removed surgically. The duodenum was cut into slices with a scalpel. Thereafter, tissue specimens were digested in RPMI 1640 supplemented with 0.1% bovine serum albumin, 1 nM selenium, antibiotics, type I collagenase (300 collagen digestion unit/nil) (Sigma-Aldrich Italy), 0.3 mg/ml deoxy-ribonuclease (Sigma-Aldrich, Italy), at 37° C. with frequent agitation for 30-45 min. Suspensions were filtered through a 800 micron metallic mesh filter (IDEALE ACLRI9 inox stainless steel) and spin at 270 g for 10 min before resuspension. Thereafter, cell suspensions were passed consecutively through 100 and 30-micron mesh filters; then, cell counting was carried-out by Fast-Read 102 (Biosigma Srl, Venice, Italy) and cell viability by the Trypan Blue assay (expressed as % of viable cells over total cells).

The same method previously developed and utilized successfully for the liver and the biliary tree, when employed in the human duodenum (N=5), resulted in the isolation of 40,816,000 (Standard Deviation) cells and only half of them were vital by Trypan Blue assays (viability 43+/−12.8%). Irrespective of the number of viable cells, isolated cells were unable to adhere and survive in culture where macro-aggregates appeared containing coated and crushed cells and leading to cell death and making it impossible to obtain cell cultures. Moreover, cultures obtained by this method were always contaminated by bacteria (5/5).

This unsuccessfully result could be due to the physical and chemical properties of the digested tissues which are highly saturated with mucus and degradation products released by mucosal epithelial cells, and that this leads to the entrapment of cells in the mucosal debris and are crushed during the procedure.

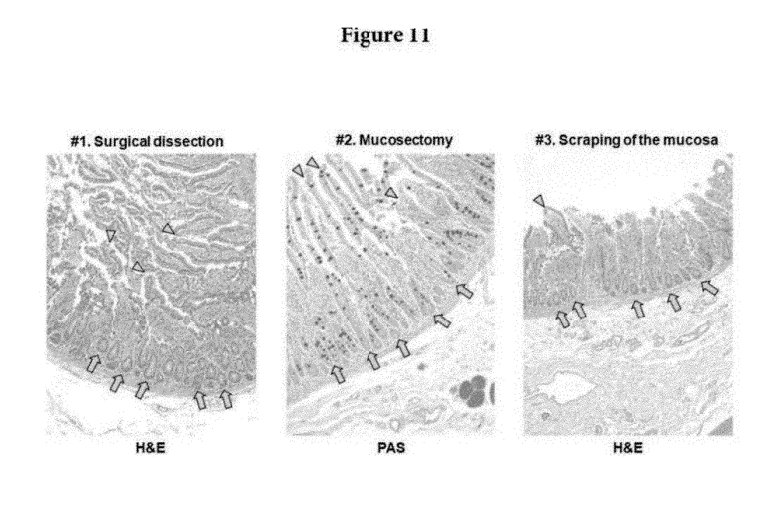

Another crucial point was the presence of the intestinal stem cell niche (intestinal crypts) within the mucosa layer. Given these findings, we elected to separate the mucosal epithelia from the submucosa. These were done by 4 different strategies: 1) surgical dissection (N=3), 2) mucosectomy after injection of normal saline under the mucosa (N=3), 3) scraping the mucosa (N=3), and 4) selective solubilization of the mucosa layer (N=10). The best strategy in term of mucosa removal was strategy #4 since strategies #1-3 resulted in partial removal of mucosa layer with the presence of intestinal crypts (FIG. 11). Moreover, the strategy #4 resulted the best approach in terms of cell isolation and viability.

Selective Solubilization of the Duodenum Mucosa by a Specific Detergent Solution Injected into the Intestinal Lumen.

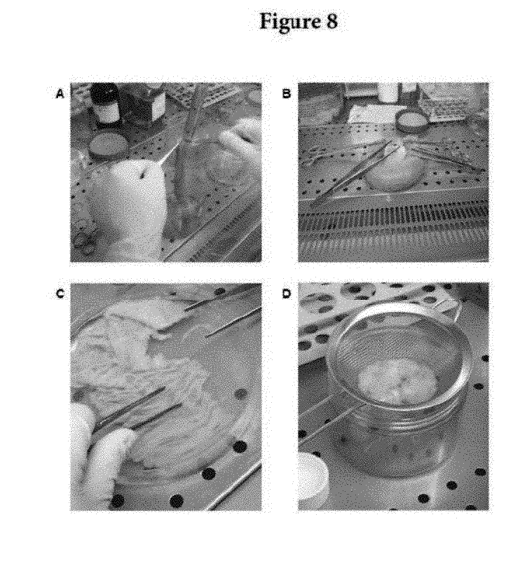

After separating the duodenum near the head of the pancreas, the ampulla of Vater was completely removed, and the intestine was closed by clamping with a surgical clamp. The extremities of the duodenum were opened; the intestinal mucus was removed by squeezing it out from the inferior incision by pressing the tissue from top to bottom. This part of the operation is very important, because the tissue should be as free from mucus as much as possible. By using a 25 ml serological pipette, approximately 200 mis of distilled water (Gibco, Italy) was flushed into the duodenum from the superior extremity incision; the inferior extremity incision was maintained clamped to collect the water (FIG. 8A). Thereafter, the intestine was kept filled completely with distilled water by clamping both extremities for about 20 minutes to induce osmotic damage selective to the mucosa epithelia cells. In this way, the duodenum will look turgid (FIG. 8B). After opening the inferior extremity and removing the water, the internal duodenum was washed twice with 100 ml of DPBS (Gibco) by utilizing 25 ml serological pipettes. By using a 25 ml serological pipette, approximately 200 ml of DPBS (Gibco, Italy) was flushed into the duodenum from the superior extremity incision. By adopting a similar procedure, the internal duodenum was filled and kept filled for 1 minute with the detergent solution (100 ml), constituted of 0.5 ml Phosphatidylcholine (Sigma-Aldrich, Italy), 20 mg Deoxycholic acid (Sigma-Aldrich, Italy), 99.5 ml of DPBS (Gibco, Italy). The solution was again removed by opening the inferior extremity, and the internal duodenum was washed with 100 ml DPBS. Finally, the duodenum was transferred into a 10 cm sterile petri dish and opened by a longitudinal incision (FIG. 8C).

A further peeling of the mucosa was obtained by using a sterile scalpel in an up/down as well as transverse direction, paying close attention to remove the mucus from the small plica (fold) of tissue. The tissue was washed in a sterile container with 100 ml DPBS (Gibco, Italy). A sterilization passage was obtained by immerging the tissue for a few seconds in 200 ml of 0.05% Sodium Hypochlorite followed by a wash via rinsing with DPBS solution. Then, the tissue was cut into small pieces by using sterile scissors and a scalpel. After the mechanical and chemical procedures to remove the mucosa, specimens were collected and processed for histo-morphological analysis. Thereafter tissue specimens were processed as previously described (FIG. 8D). Briefly, the tissue fragments were collected in two M tubes (Milteny Biotec, Germany) filled with digestion buffer and shaken. In order to reach a dissociation state of tissue, one or two cycles of the program of the MACS Dissociator (Milteniy Biotec, Germany) were performed (opacity of the solution should be noted together with yielding of very small tissue pieces). The digestion buffer was preheated to 34° C. for 10 minutes (the temperature at which the enzyme has the best efficiency). After the mechanical dissection the solution containing the tissue fragments was diluted in a solution containing DTT (Sigma-Aldrich, Italy) (see details of the composition below) and placed into two 50 ml Falcon tubes. The Falcon tubes were centrifuged for 5 minutes at 1,300 rpm (300 g). Pellets were collected and inserted into a 75 cm squared culture flask in the presence of 150 ml of digestion buffer. The flask was sealed by parafilm (Parafilm, US) and placed horizontally in a water heater at 37° C. and 5% CO₂ for about 30 minutes with shaking from time to time in order to control digestion. Thereafter the flask was placed vertically for approximately 10 minutes to let cells sediment by gravity. The floating supernatant at the surface of the solution contains impurities and can be discarded using a 10 ml serological pipette.

After the enzymatic digestion, the buffer containing the tissue fragments was placed into four 50 ml Falcon tubes. The Falcon tubes were centrifuged for 5 minutes at 1,300 rpm (300 g). The pellets were collected into two 50 ml Falcon tubes and diluted with a solution containing DTT (see details of the composition below) and then the tubes were centrifuged for 5 minutes at 1,300 rpm (300 g). The supernatants were collected and placed on an 800-micron metallic mesh filter (IDEALE ACLRI9 inox stainless steel) to be filtered with fresh cell wash. The filtrate material was collected in a sterile container. A scraper and the plunger of a syringe (Terumo #SS—20ES2) were used to facilitate passage and further dissect tissue during filtration. The resulting suspension, average of 200 ml was treated with two vials of DNase Pulmozyme 2500 U/2.5 ml (Roche, Italy) (FIG. 8D).

Isolation of BGSC from Fetal Duodenum and from Endoscopic Duodenal Biopsy Performed on Adult Subjects

The methods for isolating BGSC from duodenal biopsies and fetal organ are less complex compared to procedures optimized for obtaining cells from adult intact duodenum.

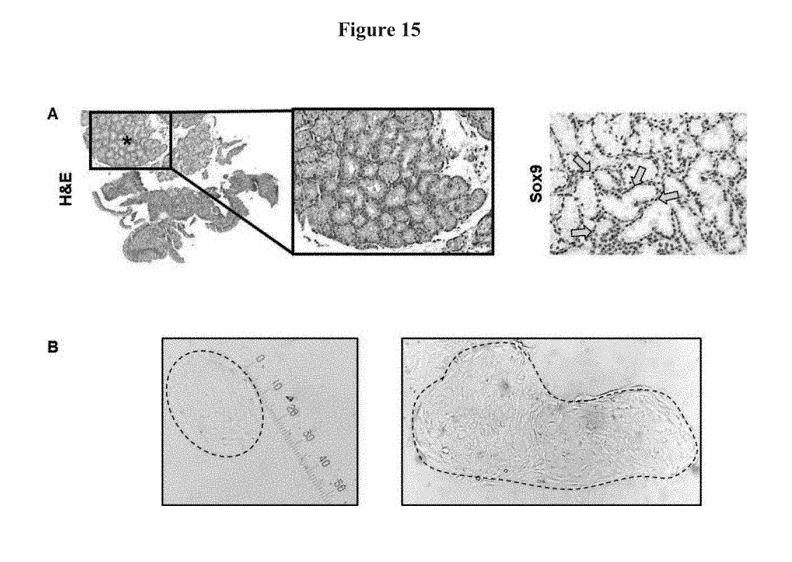

Duodenal biopsies were taken at the level of bulb and distally using forceps during gastroscopy at the Department of Translational and Precision Medicine. This method is used by trained gastroenterologists to obtain material for a wide spectrum of diseases. The procedure was done endoscopically with a flexible endoscope. The endoscope was introduced orally. The biopsies were obtained by sight, thereby avoiding any arteries or veins. The histological examination of collected biopsies revealed that BGs are present in biopsies obtained from bulb, but not from distal duodenum (FIG. 15A); BGs lie in submucosal layer just below the muscularis mucosae. Then, two to four biopsies from duodenal bulb per patient were collected during this procedure and used for isolating cells.

The fetal duodenum was harvested from fetuses (18^{th}-22^{nd} weeks: therapeutic abortion at the Department of Gynaecology and Obstetrics) by cutting proximally at the level of pylorus and distally at the level of the Treitz ligament. The pancreas and the intra-pancreatic bile duct were removed by eliminating them at the level of the hepato-pancreatic ampulla.

Subsequently, the entire fetal duodenum or the entire dudenal biopsies were further disaggregated gently by scalpel and a MACS dissociator (Miltenyi Biotec), and digested in buffer containing 300 Um′ type I Collagenase (Sigma Aldrich) and 0.3 mg/ml deoxyribonuclease (Sigma Aldrich) for 20-30 min at 37° C. Freshly isolated cells were immunoselected for TRA1-60-positive cells using magnetic beads (Miltenyi biotec).

The sorting resulted in isolation of average 12 million viable cells from fetal duodenum (N=3) and 100,000 viable cells from duodenal bulb biopsies (N=2). The duration of the isolation procedure averaged 5 hours. Cells were suspended in sterile 10% glucose solution at 1 million cells per ml and maintained for 45 minutes under controlled temperature of 4° C. before culture. Self-replicating Brunner's Gland cells according to this protocol are shown in FIG. 15B. All the procedures were carried out according to “The rules governing medicinal products in the European Union” and the European guidelines of GMP for medicinal products for human use (EudraLex—Volume 4 Good manufacturing practice Guidelines). Cell products were evaluated by standard sterility tests for gram+, gram-, aerobic and anaerobic bacteria, mycetes and with endotoxins tests, and characterized immediately by Flow Cytometry (FC) for TRA1-60 (Miltenyi Biotec, human; dilution 1:50).

### Example 2—Characterization of Tissues and Cells

Studies on Human Duodenum Tissue

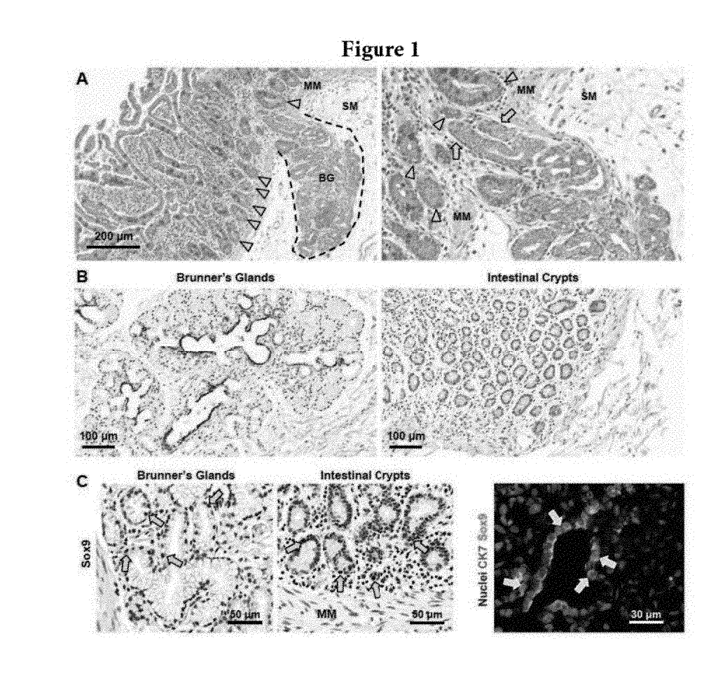

The mucosa of adult human duodenum (N=10) was elevated into intestinal villi and folded in intestinal glands (crypts) that could be transversally cut and observed deeply in the lamina propria (FIG. 1A). In the proximal portions (superior and descending) of the duodenum, the submucosa contained glandular elements (duodenal glands or Brunner's Glands: BGs) that collectively occupied 9.95±2.68% of submucosa area and 4.62±1.93% of the total wall area. BGs were composed mostly of PAS-positive mucinous cells (FIG. BGs were in anatomical continuity with intestinal crypts through the muscolaris mucosae. Few BG acini were located inside the lamina propria of the mucosa and were in continuity with intestinal crypts (FIG. 1A). In the distal portions of duodenum (inferior and ascending), BGs gradually disappeared with few glandular elements (nearly 1 per 20× field) in the inferior part and virtually no glands in the ascending portion.

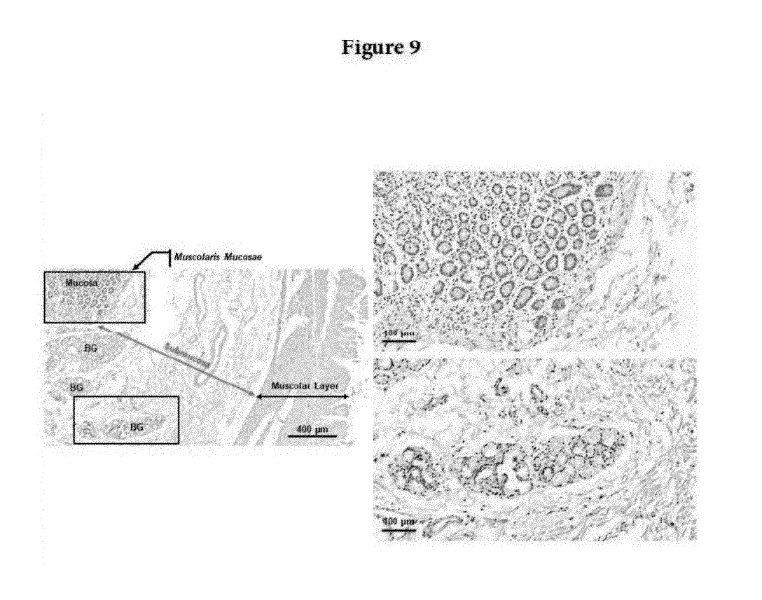

In human duodenum, the immuno-histochemical analysis indicated that BG cells and intestinal crypts partly share phenotypical traits. As regards cytokeratin expression, both intestinal crypts and BGs were CK19 positive; by contrast, CK7 was expressed specifically by some BG cells but not by intestinal glands (FIG. 1B and FIG. 9). Both intestinal glands and BGs contained cells expressing SOX9 (a marker of endodermal stem cells, FIG. 1C); in BGs, SOX9 was co-expressed with CK7 in the same cells.

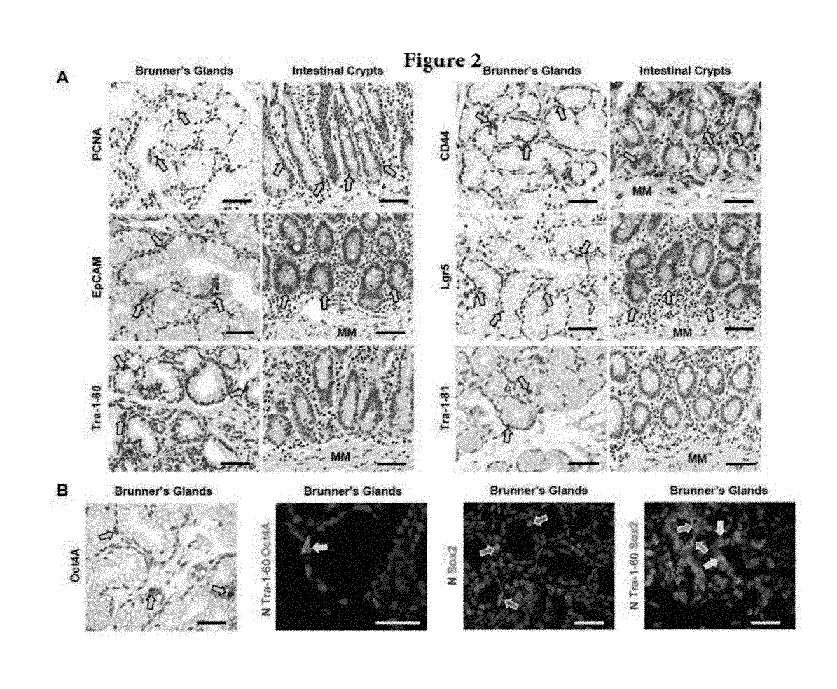

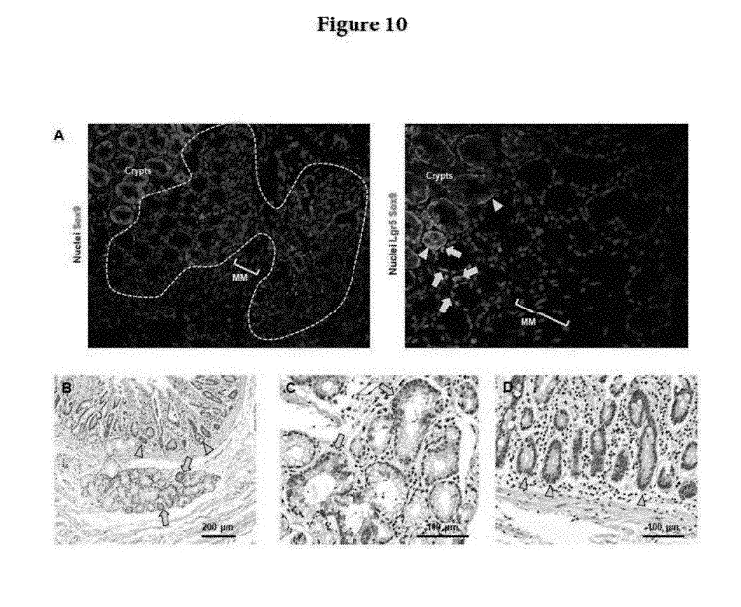

Moreover, intestinal glands and BGs contained cells expressing PCNA, a proliferation marker, and several other stem/progenitor cell markers such as CD44, EpCAM, and Lgr5 (FIG. 2A). In BGs, Lgr5 co-localized with SOX9 and its expression was greater in acini located inside the muscolaris mucosae and in continuity with intestinal crypts than in acini located deeper within the submucosal layer (FIG. 10A).

A sub-population of BG cells expressed pluripotency markers (FIG. 2). Interestingly, Tra-1-60 and Tra-1-81 were expressed by BG cells but not by cells in intestinal crypts (FIG. 2A). Tra-1-60 co-localized with SOX2 and Oct4A in the same BG cells (FIG. 2B). Finally, BGs contained cells expressing NIS which was also expressed by intestinal crypts (FIG. 10B).

In summary, the semi-quantitative analysis of stem/progenitor cell marker expression revealed that BGs comprise a niche composed of SOX9⁺ (9.12%±3.30) cells and by proliferating cells (PCNA⁺: 4.82%±1.33). Furthermore, BGs' niche contained cells expressing endodermal stem cell traits such as Lgr5 (4.76%±1.04), EpCAM (8.80%±0.65); nearly 5% of the cells expressed pluripotency markers such as Tra-1-60, Tra-1-81, Oct4A, and SOX2. Interestingly, PCNA⁺/SOX9⁺/Lgr5⁺ cells were more numerous in glandular acini in direct continuity with intestinal glands (SOX9⁺/Lgr5⁺: 11.80%±4.40; PCNA⁺: 5.95%±2.25; p<0.05) than in those located deeper in the submucosa (SOX9⁺/Lgr5⁺: 4.80%±1.50; PCNA⁺: 0.98%±0.62; p<0.05). By contrast, pluripotent cells were more numerous in acini located in a deeper position within submucosal layer.

Studies on Rodent Duodenum Tissue

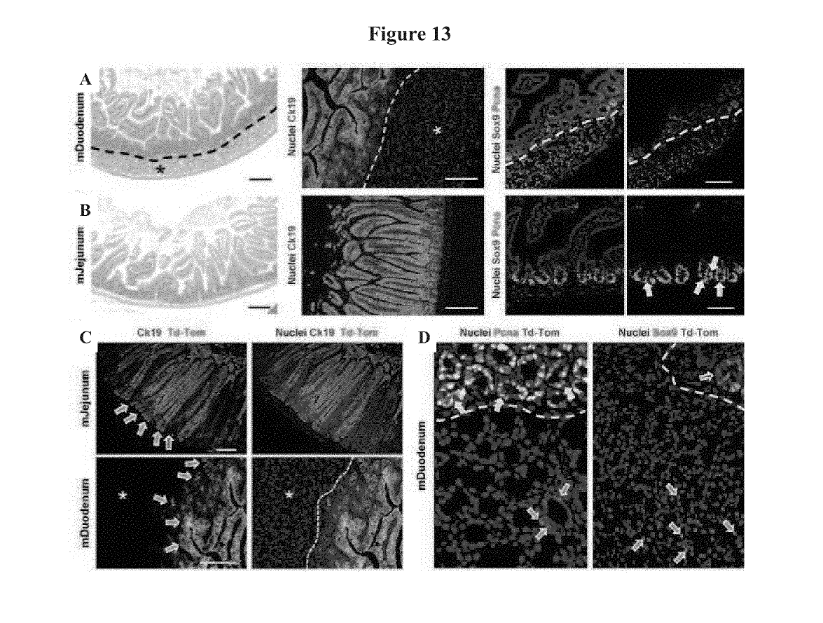

As in humans, rodent duodenum contained mucinous glands, which were located in the submucosa. Rodent SGs were in direct connection with intestinal crypts without a complete muscularis mucosae. They were distinguishable from crypts thanks to their clear cytoplasm due to mucous content (FIG. 13A). SGs were restricted to the proximal portions of rodent duodenum. When the expression of SOX9 and PCNA was studied, SOX9⁺ cells resided in SGs, while PCNA⁺ cells were mainly located in crypts (26.1±5.7%) with only a few SG cells being PCNA positive (6.7±2.2%; p<0.01 versus crypts). Duodenal SGs and crypts also differed for Ck19 expression, being the former almost negative and the latter positive (FIG. 13A). In mouse jejunum, crypts contained cells which are SOX9+, PCNA+, and Ck19+(FIG. 13B). Based on this phenotypical profile and the low percentage of PCNA+ cells within SGs, we introduced a Krt19CreTdTomatoLSL mice lineage tracing model to evaluate whether SG renewal proceeded from Ck19+/PCNA+ cells within duodenal crypts. First, jejunum was analyzed to estimate the recombination efficiency in intestinal crypts (FIG. 13C). The percentage of td-Tomato (Td-Tom) positive crypts was 72±6% with negative crypts located next to positive ones. The villi above td-Tom+ crypts resulted always in being td-Tom positive while, accordingly, the villi located just above td-Tom− crypts were td-Tom negative. When mouse duodena were examined, Ck19− SGs were almost all td6 Tom− including cells located just below 130 td-Tom+ crypts (FIG. 13C); and consistently, PCNA+ and SOX9+ cells within SGs were td-Tom− (FIG. 13D). Taken together, these data indicate that cell proliferation rate in rodent SGs is lower compared to duodenal crypts. Moreover, in physiological conditions, SG renewal is not supported by duodenal crypt cells and SOX9+ and PCNA+ cells in SGs do not derive from Ck19+ crypt cells.

Successful BGSC Isolation and Culturing Procedures

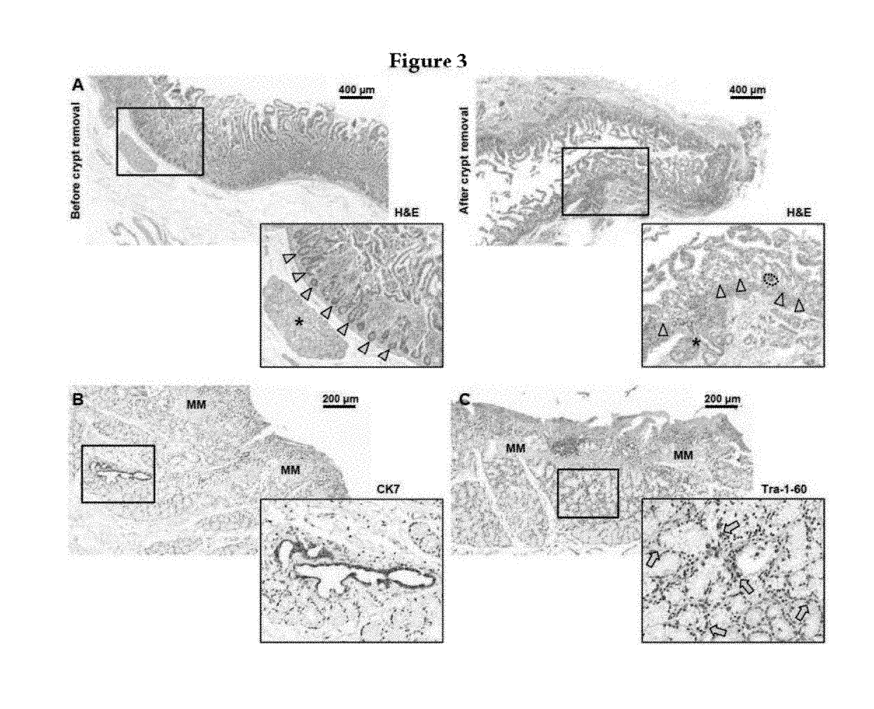

After the chemical and mechanical treatment of duodenal tissues as in Example 1, surface epithelium and almost all crypts of the mucosa layer were removed, while the connective tissue of the lamina propria and the muscolaris mucosae remained (FIG. 3A). This allowed the preservation of BGs thanks to their anatomical position below the muscolaris mucosae and within the submucosa; accordingly, BGs appeared intact and retained their CK7+(FIG. 3B), Tra-1-60⁺ (FIG. 3C), and SOX9+(not shown) cells.

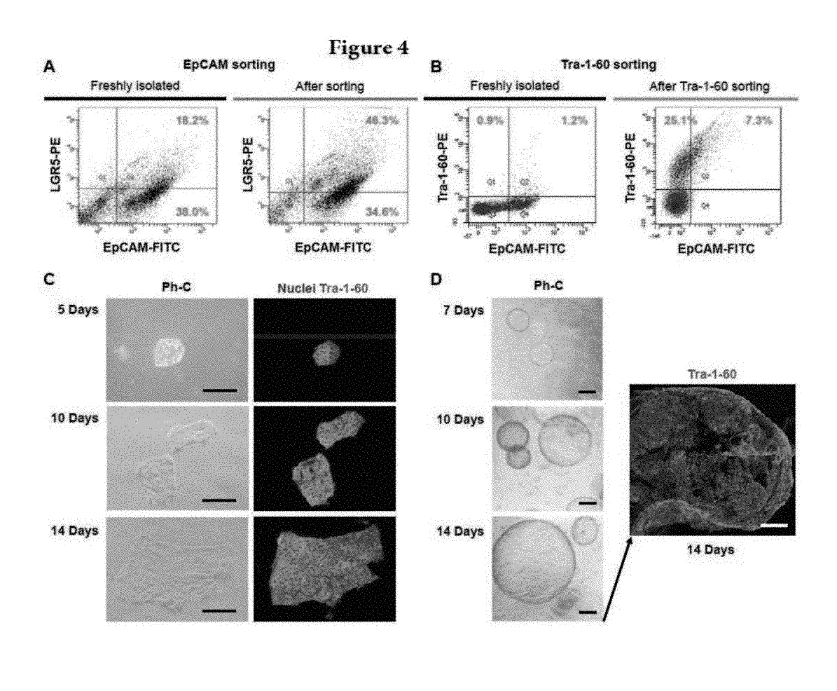

The duodenal submucosa was further processed as described in methods, and nearly 350±100 million cells were isolated with a viability >80% (85±5%). The FC showed that 40.0±18.5% of the freshly isolated cells were EpCAM⁺. When cells were immunosorted for EpCAM, the cell population was enriched to 70.3±19.3% EpCAM cells (p<0.05 vs presorting), of which 46.3%±7.3 of these cells were also Lgr5+(FIG. 4A). Cells isolated from the duodenum were also investigated by flow cytometry (FC) for Tra-1-60 expression. FC showed that 5.8±1.6% of freshly isolated cells were Tra-1-60⁺. When cells were immunosorted for Tra-1-60, the cell population was enriched to 30.4±19.8% Tra-1-60⁺ cells (p<0.05 vs presorting), and 7.3%±4.2 of the Tra-1-60⁺ cells were also EpCAM (representative scatter plot is shown in FIG. 4B). After magnetic immunosorting, the contaminating cell populations were further removed by two different culture selection approaches on plastic and as organoids. Firstly, a single cell suspension was obtained and plated at a clonal seeding density of 500 cells/cm² on plastic in serum-free Kubota's Medium, a medium allowing survival and self-replication of endodermal stem/progenitors but not of mature cells, nor of mesenchymal cells. In these conditions, only Tra-1-60⁺ cells were capable of proliferation (FIG. 4C); they started to proliferate after a 1-2 days lag period and formed small clusters of 10-15 cells after 6-8 days in culture (FIG. 4C). After 14 days, large colonies were observed (FIG. 4C). Each colony was formed mostly by small (diameter=12.06±5.76 μm), densely packed, and uniform cells with a high nucleus-to-cytoplasm ratio. Self-replication culture conditions resulted in the disappearance of almost all mesenchymal cells (not shown), as previously described in culture selection for BTSCs. Secondly, in culture conditions tailored for organoid formation, single BGSCs started to self-organize as spherical structures that further expanded in size and number. Generally, the organoids were visible after 3-5 days in culture, and their average diameters reached 2.3±5 mm within approximately 13-14 days. Organoid formation determined the enrichment of Tra-1-60k cells that represent the predominant cell phenotype forming the organoids (FIG. 4D).

Phenotypic Traits of BGSCs 2D-Colonies and BGSC-Derived Organoids

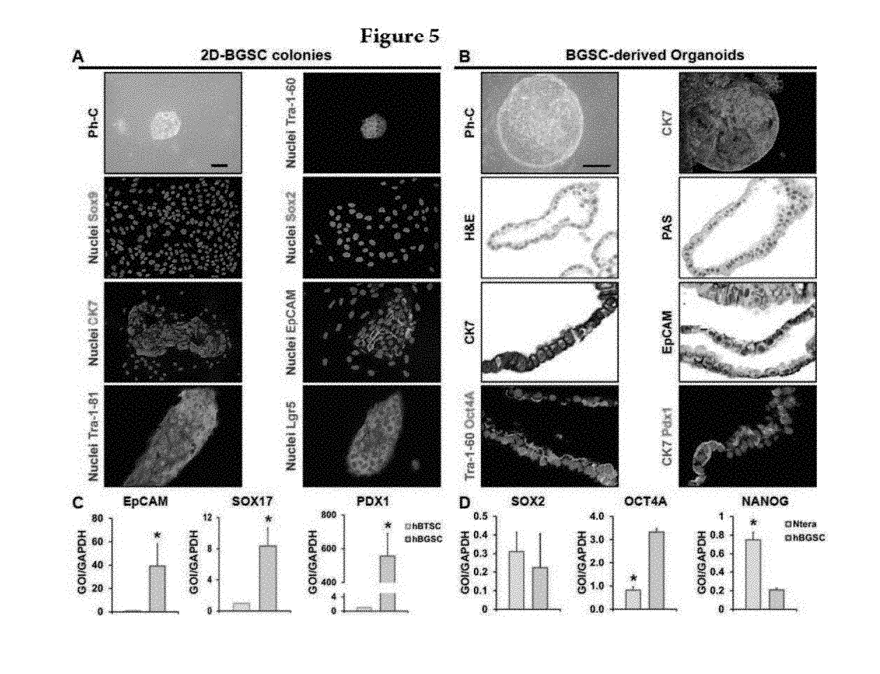

On plastic and in KM (self-replication conditions, FIG. 5A), phenotypic analysis demonstrated how cultures were composed of cells expressing CK7, SOX9, EpCAM, Lgr5, and pluripotency markers (SOX2, Tra-1-60, Tra-1-81).

In parallel, organoids (FIG. 5B) were composed of CK7⁺ and CK19⁺ cells; organoid cells expressed markers of endodermal stem cells (EpCAM, Pdxl) and pluripotency markers (Oct4A and Tra-1-60). Organoids were PAS negative (goblet cell feature) and negative for several mature cell markers such as Villin, CFTR, Albumin, Hep-Parl, and insulin (data not shown).

The BGSC phenotype has been further investigated by RT-PCR; the comparison with adequate positive controls revealed that BGSCs in monolayers on plastic and in organoids expressed biomarkers of endoderm (e.g. EpCAM, SOX17, and PDX1, FIG. 5C) and pluripotency (e.g. SOX2, OCT4A, and NANOG, FIG. 5D) genes. In these conditions, cells were mostly negative for markers of hepatocytes (i.e. albumin), cholangiocytes (i.e. CFTR), and β-pancreatic cells (i.e. insulin) lineages (data not shown).

In Vitro Differentiative Potential of BGSCs

The differentiation potential of cells isolated from BGs was evaluated by transferring them into distinct media specifically tailored to induce differentiation towards hepatic (HDM-H) or endocrine pancreatic (HDM-P) lineages.

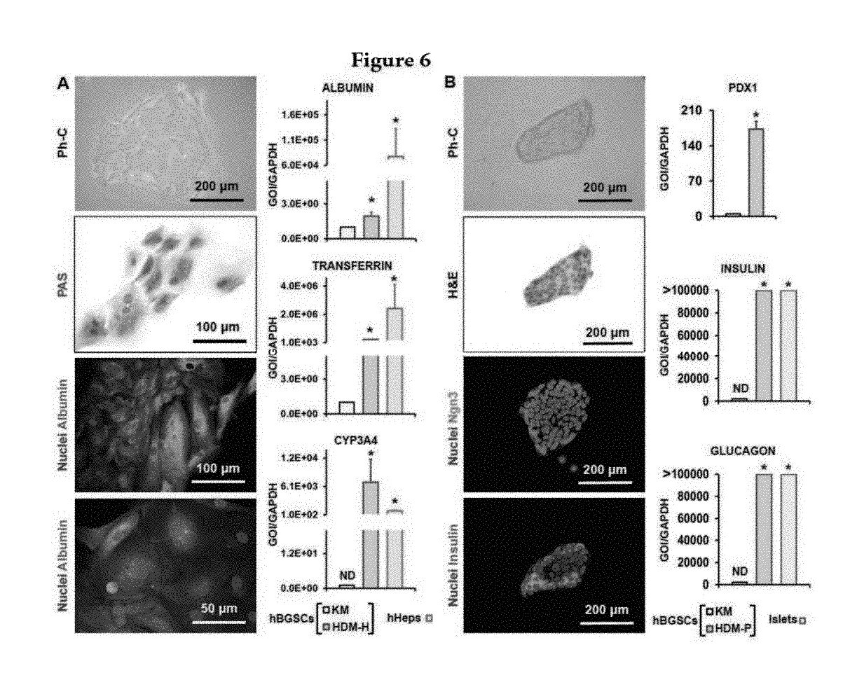

After 7 to 14 days in HDM-H (FIG. 6A), the morphology of most cells changed noticeably, from being small, spindle-shaped cells to polygonal (cuboidal)-shaped cells. These cells aggregated to form multicellular cords and had a larger diameter in comparison with cells cultured in KM (p<0.01); the cell size corresponded to that of normal, diploid, adult human hepatocytes. The IF showed that these large polygonal cells expressed albumin (FIG. 6A). Furthermore, PAS staining showed the presence of PAS-positive cells in HDM-H (FIG. 6A) but not in cells in KM (not shown) and, after digestion with α-amylase, no visible PAS staining was detectable (not shown). This supported the glycogen-storage ability of cells cultured in HDM-H. RT-PCR analysis (FIG. 6A) demonstrated that cells in HDM-H had increased expression of hepatocyte-specific genes including albumin (≈2 fold), transferrin (intermediate or zone 2; >100 fold), and CYP3A4 (drug metabolism, late or zone 3 gene; >100 fold) genes when compared with cells in KM.

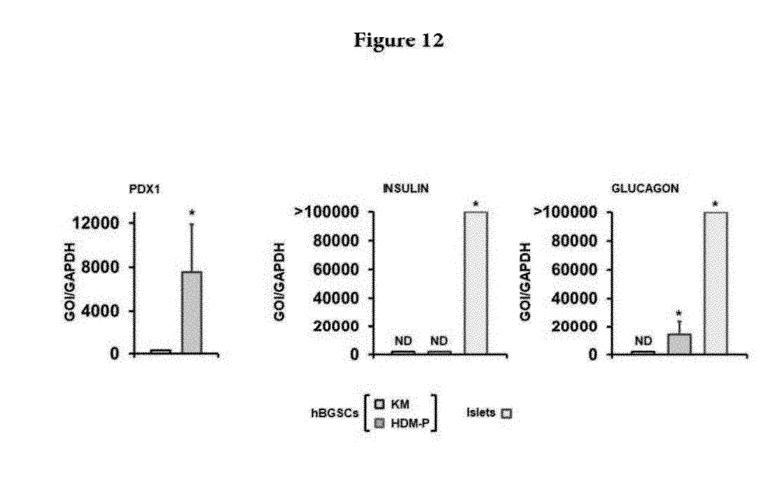

After 14 days in HDM-P, islet-like structures were observed; these structures were composed of densely packed cells expressing Ngn3 and Insulin (FIG. 6B). After 7 days in HDM-P, RT-PCR analysis demonstrated that the PDX1 but not Insulin and glucagon gene expression increased compared with the findings in cells in KM (FIG. 12). After 14 days in HDM-P, a significant increase in PDX1, insulin and glucagon gene expression was detected compared with that for cells in KM (FIG. 6B).

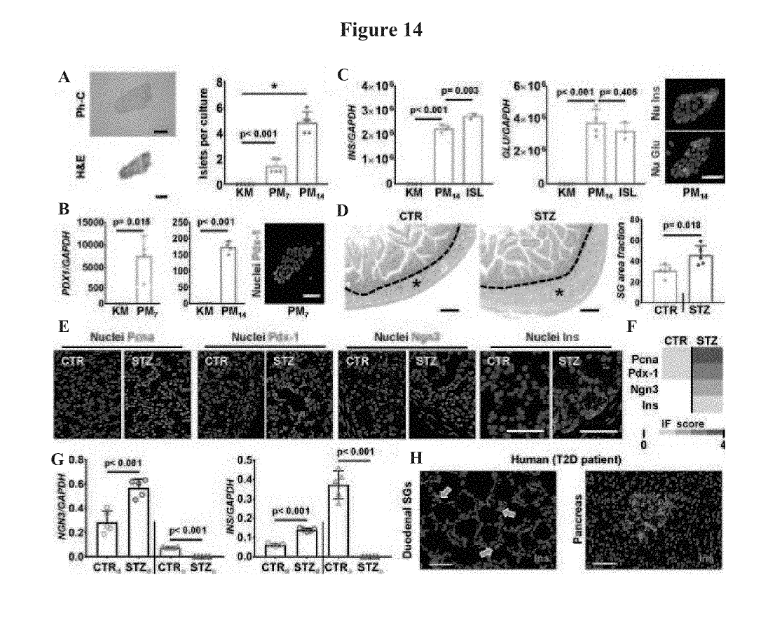

Tra-1-60+ duodenal SG cells can be rapidly restricted in vitro to β-pancreatic fate and SGs can spontaneously generate in vivo insulin expressing cells. The in vitro differentiation potency of cells isolated from duodenal SGs was evaluated by transferring them into tailored medium (PM) to induce differentiation towards endocrine pancreatic islet lineages. After 7 days in PM, the presence of rare islet-like structures was observed (1.4±0.5 per culture) (FIG. 14A) RT-PCR analysis indicated that PDX1 but not the Insulin gene was up-regulated in PM compared to KM. In parallel, islet-like structures showed expression of PDX1, but not of insulin as determined by immunofluorescence analysis (FIG. 14B). After 14 days in PM, the number of islet-like structures significantly increased compared to 7 days (4.8±0.8 per culture; p<0.01). PDX1 gene expression was higher in 14-day PM compared to KM, but lower compared to 7-day PM based on RT-PCR analysis (FIG. 14B). Insulin and Glucagon gene expressions were extremely increased at 14 days (FIG. 14C), reaching the levels of pancreatic islets used as controls. After 14 days, insulin+ and glucagon+ cells appeared in islet-like structures (FIG. 14C).

To study the potency of duodenal SG cells to commit in vivo towards endocrine pancreatic fate, we investigated whether experimentally induced diabetes in mice could trigger the acquisition of specific pancreatic traits. Thus, two different streptozotocin (STZ) models were studied. The High-Dose STZ model is characterized by a rapid increase of glycemic levels and high mortality. The Low-Dose STZ model showed a slower and less pronounced increase of glycemia and longer survival, allowing a prolonged observation time. When mice were treated with high STZ doses and sacrificed after 14 days, duodenal SG extent was increased compared to controls (FIG. 14D). Furthermore, a higher percentage of SG cells expressed PCNA, PDX1 and NGN3 compared to controls (FIG. 14E-F). Finally, in 2/5 STZ-treated mice, but not in controls, the insulin+ and glucagon+ cells was observed within duodenal SGs (FIG. 14E-F). However, no correlation was found with glycemic profile. These data were in accordance with RT-PCR analysis of specimens from rodent duodena which are characterized by an increased expression of genes related with pancreatic endocrine fate (FIG. 14G). When low STZ doses were administrated, these features did not appear (data not shown). Finally, the expression of insulin was studied in human duodena obtained from patients affected by Type-2 Diabetes (T2D). Rare (<5%) insulin+ cells could be found in duodenal SGs from T2D patients but not in ones from normal subjects (FIG. 14H).

In Vivo Transplantation of Undifferentiated Human BGSCs into Murine Livers

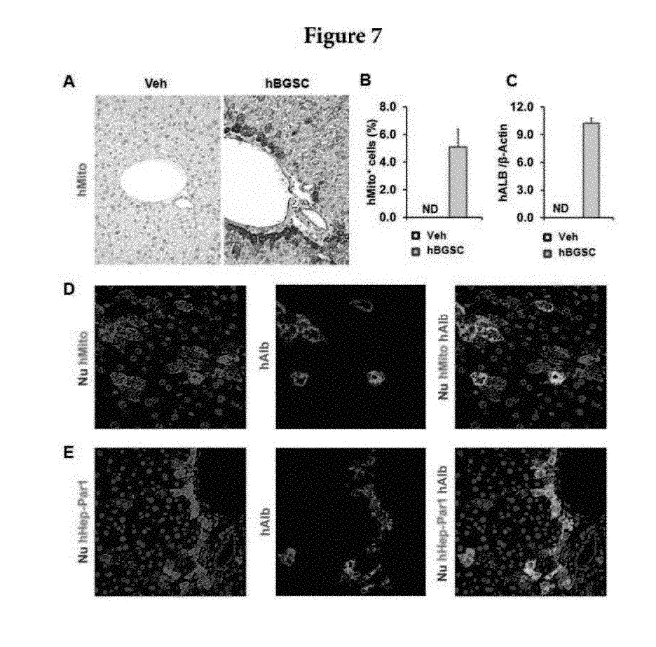

The potential of BGSC to generate in vivo mature hepatocytes was investigated by transplantation into the liver of SCID mice, via the injection through the vascular route (spleen injection). The engraftment of BGSCs was evaluated by immunostaining for specific antibodies that react only with human antigens (i.e. anti-human mitochondria, anti-human nuclei, anti-human albumin, and anti-human HepPar-1), as previously reported. One month after cell injection, human (h) mitochondrial⁺ cells were observed within murine livers (FIG. 7A); positive cells were located mostly around portal spaces, and some cells also extended towards a centrilobular position. In injected mice, nearly 5.1±1.3% of the host hepatocyte mass was represented by human antigen⁺ cells (FIG. 7B). Moreover, h-mitochondria⁺ engrafted cells were positive for mature hepatocyte markers such as albumin (FIG. 7D) and HepPar-1 (FIG. 7E). Rarely, cells positive for anti-human nuclei were observed within interlobular bile ducts. These cells were positive for CK19 (data not shown).

To confirm the effective engraftment and differentiation of transplanted hBGSCs, Applicants further investigated the human albumin mRNA expression in the murine livers. Human albumin mRNA was measurable in liver harvested from injected mice (FIG. 7C) but not detected in the sham-control mice (infused with saline).

### REFERENCES

Kubota, H. & Reid, L. M. Clonogenic hepatoblasts, common precursors for hepatocytic and biliary lineages, are lacking classical major histocompatibility complex class I antigens. Proc. Natl. Acad. Sci. (USA) 97, 12132-12137 (2000).
Furth, M. E., et al. Stem Cell Populations Giving Rise to Liver, Biliary Tree and Pancreas. in The Stem Cells Handbook, 2nd Edition (ed. Sell, S.) 75-126 (Springer Science Publishers, NY, NY, NYC, NY, 2013).
Harrill, J. A., et al. Lineage Dependent Effects of Aryl Hydrocarbon Receptor Agonists Contribute to Liver Tumorigenesis. Hepatology 61 548-560 (2015).
Lanzoni G, Cardinale V, Carpino G. The hepatic, biliary, and pancreatic network of stem/progenitor cell niches in humans: A new reference frame for disease and regeneration. Hepatology 2016; 64:277-286.
Dipaola F, Shivakumar P, Pfister J, Walters S, Sabla G, Bezerra J A. Identification of intramural epithelial networks linked to peribiliary glands that express progenitor cell markers and proliferate after injury in mice. Hepatology 2013; 58:1486-1496.
Cardinale V, Wang Y, Carpino G, Cui C B, Gatto M, Rossi M, et al. Multipotent stem/progenitor cells in human biliary tree give rise to hepatocytes, cholangiocytes, and pancreatic islets. Hepatology 2011; 54:2159-2172.
Carpino G, Cardinale V, Onori P, Franchitto A, Berloco P B, Rossi M, et al. Biliary tree stem/progenitor cells in glands of extrahepatic and intraheptic bile ducts: an anatomical in situ study yielding evidence of maturational lineages. J Anat 2012; 220:186-199.
Carpino G, Renzi A, Cardinale V, Franchitto A, Onori P, Overi D, et al. Progenitor cell niches in the human pancreatic duct system and associated pancreatic duct glands: an anatomical and immunophenotyping study. J Anat 2016; 228:474-486.
Carpino G, Cardinale V, Gentile R, Onori P, Semeraro R, Franchitto A, et al. Evidence for multipotent endodermal stem/progenitor cell populations in human gallbladder. J Hepatol 2014; 60:1194-1202.
Lanzoni G, Oikawa T, Wang Y, Cui C B, Carpino G, Cardinale V, et al. Concise review: clinical programs of stem cell therapies for liver and pancreas. Stem Cells 2013; 31:2047-2060.
Wang Y, Lanzoni G, Carpino G, Cui C B, Dominguez-Bendala J, Wauthier E, et al. Biliary tree stem cells, precursors to pancreatic committed progenitors: Evidence for possible life-long pancreatic organogenesis. Stem Cells 2013; 31:1966-1979.
Cardinale V, Wang Y, Carpino G, Mendel G, Alpini G, Gaudio E, et al. The biliary tree—a reservoir of multipotent stem cells. Nat Rev Gastroenterol Hepatol 2012; 9:231-240.
Zaret K S, Grompe M. Generation and regeneration of cells of the liver and pancreas. Science 2008; 322:1490-1494.
Jennings R E, Berry A A, Strutt J P, Gerrard D T, Hanley N A. Human pancreas development. Development 2015; 142:3126-3137.
Semeraro R, Carpino G, Cardinale V, Onori P, Gentile R, Cantafora A, et al. Multipotent stem/progenitor cells in the human foetal biliary tree. J Hepatol 2012; 57:987-994.
Broutier L, Andersson-Rolf A, Hindley C J, Boj S F, Clevers H, Koo B K, et al. Culture and establishment of self-renewing human and mouse adult liver and pancreas 3D organoids and their genetic manipulation. Nat Protoc 2016; 11:1724-1743.
Onori P, Alvaro D, Floreani A R, Mancino M G, Franchitto A, Guido M, et al. Activation of the IGF1 system characterizes cholangiocyte survival during progression of primary biliary cirrhosis. J Histochem Cytochem 2007; 55:327-334.
Carpino G, Puca R, Cardinale V, Renzi A, Scafetta G, Nevi L, et al. Peribiliary Glands as a Niche of Extrapancreatic Precursors Yielding Insulin-Producing Cells in Experimental and Human Diabetes. Stem Cells 2016; 34:1332-1342.
Della Corte C, Carpino G, De Vito R, De Stefanis C, Alisi A, Cianfarani S, et al. Docosahexanoic Acid Plus Vitamin D Treatment Improves Features of NAFLD in Children with Serum Vitamin D Deficiency: Results from a Single Centre Trial. PLoS One 2016; 11:e0168216.
Carpino G, Pastori D, Baratta F, Overi D, Labbadia G, Polimeni L, et al. PNPLA3 variant and portal/periportal histological pattern in patients with biopsy-proven non-alcoholic fatty liver disease: a possible role for oxidative stress. Sci Rep 2017; 7:15756.
Chomczynski P, Sacchi N. The single-step method of RNA isolation by acid guanidinium thiocyanate-phenol-chloroform extraction: twenty-something years on. Nat Protoc 2006; 1:581-585.
Weiss T S, Lichtenauer M, Kirchner S, Stock P, Aurich H, Christ B, et al. Hepatic progenitor cells from adult human livers for cell transplantation. Gut 2008; 57:1129-1138.
Woo D H, Kim S K, Lim H J, Heo J, Park H S, Kang G Y, et al. Direct and indirect contribution of human embryonic stem cell-derived hepatocyte-like cells to liver repair in mice. Gastroenterology 2012; 142:602-611.
Nevi L, Cardinale V, Carpino G, Costantini D, Di Matteo S, Cantafora A, et al. Cryopreservation protocol for human biliary tree stem/progenitors, hepatic and pancreatic precursors. Sci Rep 2017; 7:6080.
Huch M, Gehart H, van Boxtel R, Hamer K, Blokzijl F, Verstegen M M, et al. Long-term culture of genome-stable bipotent stem cells from adult human liver. Cell 2015; 160:299-312.
Schmelzer E, Zhang L, Bruce A, Wauthier E, Ludlow J, Yao H L, et al. Human hepatic stem cells from fetal and postnatal donors. J Exp Med 2007; 204:1973-1987.
Kajstura J, Rota M, Hall S R, Hosoda T, D'Amario D, Sanada F, et al. Evidence for human lung stem cells. N Engl J Med 2011; 364:1795-1806.
Riccio M, Carnevale G, Cardinale V, Gibellini L, De Biasi S, Pisciotta A, et al. Fas/Fas ligand apoptosis pathway underlies immunomodulatory properties of Human Biliary Tree Stem/Progenitor Cells. J Hepatol 2014; 61:1097-1105.
Hughes N R, Bhathal P S, Francis D M. Phenotypic identity of gastric mucous neck cells and mucous cells of cardiac, pyloric, and Brunner's glands. J Clin Pathol 1994; 47:53-57.
Krause W J. Brunner's glands: a structural, histochemical and pathological profile. Prog Histochem Cytochem 2000; 35:259-367.
Clevers H. The intestinal crypt, a prototype stem cell compartment. Cell 2013; 154:274-284.
Stevens M L, Chaturvedi P, Rankin S A, Macdonald M, Jagannathan S, Yukawa M, et al. Genomic integration of Wnt/beta-catenin and BMP/Smadl signaling coordinates foregut and hindgut transcriptional programs. Development 2017; 144:1283-1295.
Wang Y, Qin J, Wang S, Zhang W, Duan J, Zhang J, et al. Conversion of Human Gastric Epithelial Cells to Multipotent Endodermal Progenitors using Defined Small Molecules. Cell Stem Cell 2016; 19:449-461.
Lemaigre F P. Mechanisms of liver development: concepts for understanding liver disorders and design of novel therapies. Gastroenterology 2009; 137:62-79.
Udager A, Prakash A, Gumucio D L. Dividing the tubular gut: generation of organ boundaries at the pylorus. Prog Mol Biol Transl Sci 2010; 96:35-62.
Wandzioch E, Zaret K S. Dynamic signaling network for the specification of embryonic pancreas and liver progenitors. Science 2009; 324:1707-1710.
Burke Z D, Tosh D. Ontogenesis of hepatic and pancreatic stem cells. Stem Cell Rev 2012; 8:586-596.
Forbes S J, Gupta S, Dhawan A. Cell therapy for liver disease: From liver transplantation to cell factory. J Hepatol 2015; 62:5157-169.
Hannoun Z, Steichen C, Dianat N, Weber A, Dubart-Kupperschmitt A. The potential of induced pluripotent stem cell derived hepatocytes. J Hepatol 2016; 65:182-199.
Reid L M. Stem/progenitor cells and reprogramming (plasticity) mechanisms in liver, biliary tree, and pancreas. Hepatology 2016; 64:4-7.
Rezvani M, Grimm A A, Willenbring H. Assessing the therapeutic potential of lab-made hepatocytes. Hepatology 2016; 64:287-294.

1-64. (canceled)

65. A method of isolating one or more Brunner's Gland stem/progenitor cells (BGSCs) from a duodenum of a subject, a portion thereof, or a sample taken from same comprising:
(a) contacting a mucosal layer of a duodenum, which is substantially free of intestinal mucus, with a medium or solution having osmolality properties falling outside a physiological range under conditions that induce osmotic shock to the cells of the mucosal layer;
(b) removing or dissolving at least a portion of the mucosal layer or the cells thereof by mechanical, surgical and/or chemical methods, leaving and/or exposing a remainder which may include a submucosal layer;
(d) digesting or dissociating the remainder; and
(e) isolating one or more BGSCs from the digested remainder.

66. The method of claim 65 in which the isolating step comprises isolating BGSCs that express one or more markers selected from the group Tra-1-60, Tra-1-81, OCT4, SOX2, NANOG, EpCAM, SOX9, CK7, Lgr5, NIS, CD44 and CK19.

67. The method of claim 65 in which the isolating step comprises isolating BGSCs that express both SOX17 and PDX1.

68. The method of claim 65 in which the duodenum tissue is rendered substantially free of intestinal mucus, optionally by squeezing the duodenum tissue.

69. The method of claim 65 in which the medium or solution having osmolality properties falling outside a physiological range comprises a hypotonic, hypoosmotic, hypertonic, or hyperosmotic solution.

70. The method of claim 65 in which the medium having osmolality properties falling outside a physiological range comprises a glucose solution, a high salt solution, or distilled water.

71. The method of claim 65 in which removal of at least a portion of the mucosal layer or the cells thereof is carried out by chemical disruption, which comprises a use of an emulsifier and/or a detergent.

72. The method of claim 65 in which the detergent and/or emulsifier is in water, saline and/or a buffer.

73. The method of claim 65 in which the detergent and/or emulsifier is applied for a period of less than 15 minutes.

74. The method of claim 65 in which the emulsifier is selected from a group comprising Lecithin, Polyoxyethylene Sorbitan Monolaurate (Polysorbate 20), Polyoxyethylene Sorbitan Monooleate (Polysorbate 80), Polyoxyethylene Sorbitan Monopalmitate (Polysorbate 40), Polyoxyethylene Sorbitan Monostearate (Polysorbate 60), Polyoxyethylene Sorbitan Tristearate (Polysorbate 65), Ammonium Phosphatides, Sodium, Potassium and Calcium Salts of Fatty Acids, Magnesium Salts of Fatty Acids, Mono- and Diglycerides of Fatty Acids, Acetic Acid Esters of Mono- and Diglycerides of Fatty Acids, Lactic Acid Esters of Mono- and Diglycerides of Fatty Acids, Citric Acid Esters of Mono- and Diglycerides of Fatty Acids, Mono- and Diacetyl Tartaric Acid Esters of Mono- and Diglycerides of Fatty Acids, Mixed Acetic and Tartaric Acid Esters of Mono- and Diglycerides of Fatty Acids, Sucrose Esters of Fatty Acids, Sucroglycerides, Polyglycerol Esters of Fatty Acids, Polyglycerol Polyricinoleate, Propane-1,2-Diol Esters of Fatty Acids, Thermally Oxidised Soya Bean Oil Interacted with Mono- and Diglycerides of Fatty Acids, Sodium Stearoyl-2-Lactylate, Calcium Stearoyl-2-Lactylate, Sorbitan Monostearate, Sorbitan Tristearate, Sorbitan Monolaurate, Sorbitan Monooleate, Sorbitan Monopalmitate and combinations thereof.

75. The method of claim 65 in which the detergent is selected from a group comprising 1-Heptanesulfonic Acid; N-Laurylsarcosine, Lauryl Sulfate, 1-Octane Sulfonic Acid and Taurocholic Acid, Benzalkonium Chloride, Cetylpyridinium, Methylbenzethonium Chloride, Decamethonium Bromide, Alkyl Betaines, Alkyl Amidoalkyl Betaines, N-Dodecyl-N,N-Dimethyl-3-Ammonio-1-Propanesulfonate, Phosphatidylcholine, N-Decyl A-D-Glucopyranoside, N-Decyl A-D-Maltopyranoside, N-Dodecyl B-D-Maltoside, N-Octyl B-D-Glucopyranoside, N-Tetradecyl B-D-Maltoside, Tritons (Triton X-100), Nonidet-P-40, Poloxamer 188, Sodium Lauryl Sulfate, Sodium Deoxycholate, Sodium Dodecyl Sulfate and combinations thereof.

76. The method of claim 65 in which the remainder comprises a submucosal layer.

77. The method of claim 65 in which digestion or dissociation is carried out enzymatically.

78. The method of claim 65 in which the tissue sample is minced before the digestion or dissociation step.

79. The method of claim 65 in which the digestion or dissociation step and/or the isolation step is performed in low attachment plates.

80. The method of claim 65 in which the isolation step is performed using culture selection with culture conditions that comprise a serum-free medium, optionally, Kubota's Medium.

81. The method of claim 65 in which the isolation step is performed using culture selection with culture conditions that comprise a medium containing serum.

82. The method of claim 6 in which the isolated cells are cultured under conditions that support or produce spheroids, one or more organoids, cell clusters, or cell aggregates.

83. A method of isolating one or more BGSCs from a duodenum of a subject, a portion thereof, or a sample taken from same, comprising the following steps, in which the step to substantially kill, inactivate, or remove pathogens and/or pathogenic and/or beneficial microbes can be carried out at any time or more than once:
(a) removing the intestinal mucus;
(b) applying a medium or solution having osmolality properties falling outside a physiological range under conditions that induce osmotic shock to the cells of the mucosal layer;
(c) removing or dissolving at least a portion of the mucosal layer or the cells thereof by mechanical, surgical and/or chemical methods, leaving and/or exposing a remainder which may include a submucosal layer;
(d) applying to the mucosal layer and/or the remainder a medium or solution to substantially kill, inactivate or remove pathogens and/or pathogenic and/or beneficial microbes;
(e) applying digestion or dissociation to the submucosal layer to produce a digest, dissociated cellular material, or a cell suspension;
(f) optionally culturing at least some of the digest, dissociated cellular material, or cells from the cell suspension; and
(g) isolating those cells that express one or more of Tra-1-60, Tra-1-81, OCT4, SOX2, NANOG, EpCAM, SOX9, CK7, Lgr5, NIS, CD44, CK19; and/or those cells that express both SOX17 and PDX1.

84-92. (canceled)

93. The method of claim 83 in which the medium or solution to substantially kill, inactivate or remove pathogens and/or pathogenic and/or beneficial microbes comprises an aqueous solution of sodium hypochlorite (NaClO) or any solution(s) or agent(s) used for disinfection of skin or surfaces.

94. The method of claim 83 in which application of a medium or solution to substantially kill, inactivate or remove pathogens and/or pathogenic and/or beneficial microbes takes place before the application of the detergent and/or emulsifier, or takes place after the digestion or dissociation, or takes place after the removal of mucus.

95-113. (canceled)

114. A method of isolating BGSCs from a duodenum of a subject, a portion thereof, or a sample taken from same comprising:
(a) digesting or dissociating a duodenum, a portion thereof, or a sample taken from same to provide a digest or dissociated cellular material;
(b) obtaining from the digest or dissociated cellular material: (i) those cells that express one or more of Tra-1-60, Tra-1-81, OCT4, SOX2, NANOG, EpCAM, SOX9, CK7, Lgr5, NIS, CD44 and CK19; and/or (ii) those cells that express both SOX17 and PDX1.

115. The method of claim 114 in which the duodenum, a portion thereof, a sample taken from same, the digest, the dissociated cellular material, or combinations thereof, are contacted with a medium or solution to substantially kill, inactivate or remove pathogens and/or pathogenic and/or beneficial microbes.

116. A method of isolating one or more multipotent cells expressing one or more desired biomarkers, or a population of cells in which at least some, or a substantial portion of, or a majority of the cells express one or more desired biomarkers, from a tissue (or portion or sample thereof) having a mucosal layer and a submucosal layer, comprising the following steps, which may occur in the following sequence or may occur in a different sequence:
(a) contacting a mucosal layer of a tissue having a mucosal layer and a submucosal layer with a medium or solution having osmolality properties falling outside a physiological range under conditions that induce osmotic shock to the cells of the mucosal layer;
(b) removing or dissolving at least a portion of the mucosal layer or the cells thereof by mechanical, surgical and/or chemical methods, leaving and/or exposing a remainder, which may include a submucosal layer;
(c) contacting the remainder with a medium or solution to substantially kill, inactivate, or remove pathogens and/or pathogenic and/or beneficial microbes;
(d) digesting or dissociating the remainder;
(e) isolating one or more multipotent cells, or a population of cells in which at least some, or a substantial portion of, or a majority of the cells express one or more desired biomarkers.

117. The method of claim 116, which further comprises removing surface mucus.

118-134. (canceled)

135. The method of claim 116, which further comprises one or more wash steps using a physiologically acceptable medium.

136. The method of claim 116 in which the tissue is an endodermal tissue.

137. The method of claim 116 in which the tissue is selected from a group comprising trachea, main bronchus, esophagus, stomach, duodenum, small intestine, large intestine and rectum.

138. The method of claim 116 in which the tissue is selected from a group comprising liver, pancreas, gall bladder and biliary tree ducts, wherein the biliary tree ducts comprise common ducts and cystic ducts.
